(19) United States
(12) Patent Application Publication
(10) Pub. No.: US 2020/0246441 A1
(43) Pub. Date: Aug. 6, 2020

(54) COMPOSITIONS AND METHODS FOR AUTOIMMUNE DISEASE TREATMENT

(71) Applicants: AMRITA VISHWA VIDYAPEETHAM, Kochi (IN); MONASH UNIVERSITY, Clayton (AU)

(72) Inventors: MANZOOR KOYAKUTTY, Kochi (IN); CLAUDE C.A. BERNARD, Clayton (AU); KRISHNAKUMAR N MENON, Kochi (IN); PRASHANT SADANANDAN, Kochi (IN); NATALIE L PAYNE, Clayton (AU); SHANTIKUMAR V NAIR, Kochi (IN)

(21) Appl. No.: 16/326,536

(22) PCT Filed: Aug. 21, 2017
(86) PCT No.: PCT/US17/47778

§ 371 Date: Feb. 19, 2019

(30) Foreign Application Priority Data
Aug. 19, 2016 (AU) 2016903294

(51) Int. Cl. A61K 39/00; A61K 9/51; A61P 37/00

(52) U.S. Cl. CPC A61K 39/0008; A61P 37/00; A61K 9/5115

(57) ABSTRACT

The present invention relates to compositions and methods for the treatment or prevention of autoimmune diseases such as demyelination disorders. The invention includes a composition comprising a peptide antigen comprising at least one beta (β) amino acid coupled to a carrier nano or microparticle of silica. In one embodiment the demyelination disorder is multiple sclerosis.

### RELATED APPLICATION

This application claims benefit and priority to Australian Provisional Patent Application no: 2016903294, entitled “Compositions and methods for autoimmune disease treatment”, filed on Aug. 19, 2016. The disclosure of that Australia application is incorporated herein by reference for all purposes.

### BACKGROUND

Present disclosure generally relates to compositions and methods of treatment for autoimmune diseases. Autoimmune diseases are caused by an abnormal immune response involving either cells or antibodies directed against normal tissues. Based on the type of immune response (or immune reaction) involved, autoimmune diseases in mammals can generally be classified in one of two different categories: cell-mediated (i.e., T-cell-mediated) or antibody-mediated (i.e., B-cell mediated) disorders.

A group of autoimmune diseases are demyelination disorders. Neuronal demyelination is a deleterious condition characterized by a reduction of myelin protein in the nervous system. Myelin is a vital component of the central (CNS) and peripheral (PNS) nervous system, which encases the axons of neurons and forms an insulating layer known as the myelin sheath. The presence of the myelin sheath enhances the speed and integrity of nerve signal in form of electric potential propagating down the neural axon. The loss of myelin sheath produces significant impairment in sensory, motor and other types of functioning as nerve signals reach their targets either too slowly, asynchronously (for example, when some axons in a nerve conduct faster than others), intermittently (for example, when conduction is impaired only at high frequencies), or not at all.

A common demyelination disorder is multiple sclerosis (MS). MS is a chronic inflammatory disease of the CNS characterized by inflammation, sharply demarcated areas of demyelination and axonal loss/damage resulting in a multiplicity of neurological deficits.

MS patients generally experience one of four clinical courses of disease, each of which might be mild, moderate, or severe: relapsing-remitting, primary progressive, secondary progressive, or primary-relapsing. About 85% of MS patients have the relapsing remitting form of the disease, in which they experience clearly defined relapses (also called flare-ups or exacerbations), which are episodes of acute worsening of neurologic function, followed by partial or complete recovery periods (remissions) that are free of disease progression.

The etiology of MS is as yet unknown but it is generally accepted that the disease is the result of an autoimmune response against CNS antigens in genetically susceptible individuals. Immunological, immunohistochemical and molecular analyses of MS tissue suggest that the development of this disease is driven by a Th1+Th17-type inflammatory response, in concert with an autoantibody reaction directed against defined CNS myelin and possibly neuronal components. To date, MS has been regarded as a primary demyelinating disorder and much effort has been devoted to investigate the relationship between the evolution of the lesions and clinical progression in terms of myelin destruction and repair. It has now become apparent that axonal damage is an early event during the development of lesion formation in both MS and experimental autoimmune encephalomyelitis (EAE) and is the main arbiter of permanent clinical disability.

MS is characterized by infiltration of immune cells, localized myelin destruction, loss of oligodendrocytes and axonal degeneration. Peripheral autoreactive CD4+ T-cells specific for CNS antigens are thought to be the major purveyors of MS pathogenesis. CD4+ T cell activation is initiated when the T-cell receptor (TCR) on a T-cell recognizes a peptide presented on the surface of major histocompatibility complex (MHC) class II molecules and, in combination with additional receptor-ligand interactions, induces their activation, proliferation and differentiation into T-cells with effector function.

Disease onset usually occurs in young adults between the ages of 20 and 40 and there is currently no cure for this disabling disease and treatment is primarily focused on management of symptoms.

Till now, the majority of therapies for MS approved by the US FDA such as beta interferons and other similar drugs for autoimmune disease have focused on the global inhibition of immune inflammatory activity and it is only partially effective. Moreover, the drugs used to suppress the autoimmune response have numerous side effects. Since the current treatment options for autoimmune diseases involves nonspecific immunosuppression, immune tolerance targeted therapy is highly desired for the treatment and/or prevention of demyelination disorders, in particular multiple sclerosis. In this respect, current therapies are aimed at targeting both T- and B-cell response leading to modulation of the immune attack.

It has been proposed that coupling of antigens to leukocytes lead to suppression of the disease in EAE, the animal model of MS (Getts et al. J Immunol. 2011, 187(5), 2405-17). Another study has suggested that Proteolipid Protein (PLP) antigen coated polystyrene nanoparticle ranging to the size of leukocytes is efficient in suppressing the disease due to the uptake of antigen by splenic macrophages (Getts, et al. Nature Biotech. 2012, 30 (12), 1217-1225. Application No: PCT/US2011/060537). Further, similar effects were shown with PLP antigen coated biodegradable PLGA nanoparticles (Hunter et al. 2014, ACS nano, 8(3), 2148-2160. Application No: PCT/US2013/047079) which were coinciding with the disease suppressive effects of antigenic peptide coated leukocytes and polystyrene particles.

Despite experimental evidence demonstrating their potential for treatment of autoimmune diseases, the development of peptide-based vaccines has been hampered by their poor stability, bioavailability and rapid modification in vivo. The development of peptide-based therapeutics is problematic as peptides are rapidly cleared by the kidneys and/or inactivated by proteases in the blood. In mice this can be somewhat overcome by using Freund's Complete adjuvant which acts as an emulsion, carrying the peptide around the body as a large complex, delaying clearance. However, this approach is not feasible for use in humans.

Altered peptide ligands possess promising immunoregulatory potential for treating the animal model of MS. However, their instability and poor bioavailability is a major impediment for their clinical use (Webb et al. 2005, J. Immunol., 175, 3810-3818). One such altered therapeutic peptide is MOG 44βF, which when administered as free peptide suppressed EAE only at a very high dosage (800 μg) (Courtney et al. 2014, Journal of Neuroimmunology, 277, 1, 67-76).

At present there is no means of estimating the concentration of antigens delivered to the macrophages in secondary lymphoid organs. This dosimetry is critical as treatment based on engaging macrophages in liver and spleen for immunosupression needs to be controlled for not compromising its routine functions such as immunity against infections or viruses. Furthermore, there remains a need for a carrier material which can be visualized in the target organs such that dose can be adjusted accordingly and is easily biodegradable so that repeated dosing is possible without toxicity issues. An imageable, biodegradable, immunomodulatory nano/micro drug is currently not available.

### SUMMARY

Described herein are compositions and methods for autoimmune disease treatment. Other aspects of the invention including the corresponding peptides, nano- or micro-particle carriers, methods, and compositions are described herein.

The present invention provides a composition comprising a modified peptide antigen with a beta (β) amino acid coupled to, complexed to, or encapsulated with an imageable carrier nano- or micro-particle of silica. The composition is useful in treating multiple sclerosis and tolerating a subject to an autoimmune antigen. In some embodiments, the β amino acid is present or introduced at a position in the peptide antigen where the homologous α (alpha) amino acid in the peptide antigen interacts with a T cell receptor or MHC molecule. In one embodiment, the MHC molecule is a class II molecule. In one embodiment, the nanoparticles are configured to be specifically phagocytosed by macrophages in liver, spleen and lymph nodes, and imagable by non-invasive method such as magnetic resonance imaging. In some embodiments, the modified peptide antigen causes immunosuppression in autoreactive disease models at concentration of 10-50 times lower compared to corresponding free peptide. In some embodiments, the peptide antigen is derived from myelin oligodendrocyte glycoprotein (MOG), myelin basic protein (MBP) or proteolipid protein (PLP).

The present invention provides a composition comprising a myelin oligodendrocyte glycoprotein (MOG) peptide coupled to or encapsulated into a carrier particle, wherein the peptide comprises an amino acid sequence having at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 1, wherein the amino acid residue at, or equivalent to: position 38 in SEQ ID NO: 1 is not an αGly: position 39 in SEQ ID NO: 1 is not an αLys position 40 in SEQ ID NO: 1 is not an αTyr; position 41 in SEQ ID NO: 1 is not an αArg: position 43 in SEQ ID NO: 1 is not an αPro; position 44 in SEQ ID NO: 1 is not an αPhe; position 45 in SEQ ID NO: 1 is not an αSer: position 45 in SEQ ID NO: 1 is not an αSer; position 45 in SEQ ID NO: 1 is not an αSer; position 47 in SEQ ID NO: 1 is not an αVal: and/or position 48 in SEQ ID NO: 1 is not an αVal. In various embodiments, the amino acid residue is a non-conservative substitution relative to the amino acid that occurs in that position in SEQ ID NO: 1. In one embodiment, the amino acid is alanine or a β amino acid, or a β amino acid version of the amino acid that occurs at that position in SEQ ID NO: 1. In one embodiment, the amino acid at position 44 is an α-alanine, β-phenylalanine or β-alanine.

The present invention provides a composition comprising a myelin oligodendrocyte glycoprotein (MOG) peptide coupled to a carrier particle, wherein the peptide comprises an amino acid sequence having at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 7, wherein the amino acid residue at, or equivalent to position 119 in SEQ ID NO: 7 is not an αPhe. In various embodiments, the amino acid residue is a non-conservative substitution relative to the amino acid that occurs in that position in SEQ ID NO: 7. In one embodiment, the amino acid is alanine or a β amino acid, or a (amino acid version of the amino acid that occurs at that position in SEQ ID NO: 7. In one embodiment, the amino acid at position 119 is an α-alanine, β-phenylalanine or β-alanine. In one embodiment, provided herein is a composition comprising a peptide having a myelin oligodendrocyte glycoprotein (MOG) peptide having a (amino acid at a position, or position equivalent to, 119 in SEQ ID NO: 7 (numbering wherein Phe at the N-terminus is position 119 and Leu at the C-terminus is position 132), the peptide coupled to a carrier particle.

In some embodiments, the carrier particle comprises solid or porous silica and the particle diameter is 10-5000 nm. In some embodiments, the carrier particle is formed by solid or porous silica of spherical, rectangular, triangular, needular, prismatic, rod shape, or any combination thereof. In other embodiments, the carrier particle is doped with Fe, Gd or Mn at a concentration of 0.1-50 wt % with respect to Si. In yet other embodiments, the minimum weight ratio of peptide to nanoparticle or microparticle that is critical for the specific uptake in liver, spleen and lymph node residing macrophage is about 0.01. In one embodiment, the optimum concentration of peptide-nanoparticle conjugation and macrophage uptake, the cells produce reduced levels of one of the main pro-inflammatory cytokine, IL-17, that causes autoreactivity. In one embodiment, the carrier particle is carboxy functionalized by EDTA (Ethylene diamine tetra acetic acid), DTPA (Diethylene triamine penta acetic acid), Succinic acid, malonic acid, glutaric acid, adipic acid, pimelic acid or hyaluronic acid. In another embodiment, the carrier particle is amino functionalized by amino propyl triethoxy silanes, amino propyl trimethoxy silane, polyethylene imine, protamine sulfate, poly-L-Lysine or arginine. In yet another embodiment, the carboxy functionalized carrier particle is attached to a linker made of amino group provided by amino propyl triethoxy silanes, amino propyl trimethoxy silane, polyethylene imine, protamine sulfate, poly-L-Lysine or arginine. In some embodiments, the amino functionalized carrier particle is attached to a linker made of carboxyl group provided by EDTA, DTPA, Succinic acid, malonic acid, glutaric acid, adipic acid, pimelic acid or hyaluronic acid by forming an amide bond, or by electrostatic interactions.

The present invention also provides a method of treating, preventing, delaying the onset, or reducing the severity of an autoimmune disease in a subject in need thereof, the method comprising administering a composition of the invention as described herein to a subject identified as having an autoimmune disease thereby treating the autoimmune disease in the subject. In one embodiment, the subject is identified as being in risk of developing the autoimmune disease. In some embodiments, the autoimmune disease is a demyelination disorder, for example multiple sclerosis. In one embodiment, the multiple sclerosis is primary progressive multiple sclerosis (PPMS), relapsing-remitting multiple sclerosis (RRMS), secondary progressive multiple sclerosis (SPMS), or progressive relapsing multiple sclerosis (PRMS). In one embodiment, the nanoparticles are configured to be specifically phagocytosed by macrophages in liver, spleen and lymph nodes, and imagable by non-invasive method such as magnetic resonance imaging.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1A illustrates SEM image of 200 nm SiO₂ nanoparticles (NPs).

FIG. 1B illustrates TEM image of 200 nm SiO₂ NPs clearly showing porosity.

FIG. 1C illustrates SEM image of 500 nm SiO₂ NPs.

FIG. 1D illustrates TEM image of 500 nm SiO₂ NPs showing porosity and irregular surface.

FIG. 1E illustrates TEM image of MOG peptide conjugated 200 nm SiO₂ NPs.

FIG. 1F illustrates TEM image of MOG peptide conjugated 500 nm SiO₂ NPs.

FIG. 2A illustrates SEM image of Fe doped peptide fluorescent SiO₂ NPs.

FIG. 2B illustrates TEM image of Fe doped peptide fluorescent SiO₂ NPs.

FIG. 2C illustrates T₂ weighted MR signal intensity of Fe doped SiO₂ NPs with different iron concentrations.

FIG. 2D illustrates fluorescent microscopy image of Fe doped peptide conjugated fluorescent (FITC) SiO₂ NPs

FIG. 3A illustrates SEM image of peptide NPs uptake in vitro in mouse Raw 264.7 macrophage cell line (5×103 cells, 100 ug/mL NPs, 12 h incubation) showing less uptake of thickly coated peptide NPs (on the right, the typical TEM image of a thick peptide coated NP).

FIG. 3B illustrates SEM image of peptide NPs uptake in vitro in mouse Raw 264.7 macrophage cell line (5×103 cells, 100 ug/mL NPs, 12 h incubation) showing moderate uptake of thin peptide coated NPs (on the right, the typical TEM image of a thin peptide coated NP).

FIG. 3C illustrates SEM image of bare NPs uptake in vitro in mouse Raw 264.7 macrophage cell line (5×103 cells, 100 μg/mL NPs, 12 h incubation) showing high uptake bare NPs (on the right, the typical TEM image of a bare NP).

FIG. 3D illustrates uptake study by Prussian blue staining indicating cellular uptake of large concentration of NPs seen as blue.

FIG. 3E illustrates uptake study by Fluorescence imaging showing uptake of NPs seen as green (Nucleus-blue, DAPI stained).

FIG. 4A illustrates T2 weighted images of whole body axial MRI of C57BL/6 mice showing the accumulation of Fe doped peptide SiO₂ NPs in spleen (indicated by white arrow) i) before intravenous tail injection, ii) 4.5 h after intravenous tail vein injection showing enhanced dark T2 contrast (white arrow).

FIG. 4B illustrates T1 weighted transverse ex vivo MRI images of i) untreated mice spleen, ii) spleen of mice taken 4.5 hrs after intravenous injection, iii) untreated mice liver, iv) Liver of mice taken 4.5 hrs after intravenous injection.

FIG. 4C illustrates fluorescent microscopic image of resected spleen taken 4.5 h after peptide conjugated SiO₂ NPs injection showing an enhanced accumulation of NPs (green fluorescence) in the spleen in a concentric pattern.

FIG. 4D illustrates fluorescence images of resected liver sections taken 4.5 h after peptide conjugated SiO₂ NPs injection. (Fluorescence from NPs accumulated in the liver macrophages can be seen as green fluorescence. NPs uptake by hepatocytes are found negligible. Sections were counter stained with DAPI and TRITC Phalloidin).

FIG. 5 illustrates nanoparticles coated with MOG37-52 that stimulate the proliferation of MOG-specific T-cells. Spleen cells from MOG T-cell receptor transgenic (2D2) mice were cultured with increasing concentrations of MOG37-52 peptide or 500 nm silica nanoparticles coated with MOG37-52, MOG443F or scrambled MOG37-52. Proliferation was measured by 3H-thymidine incorporation. T-cell proliferation induced by nanoparticles coated with MOG37-52 peptide is comparable to the MOG37-52 peptide, while nanoparticles coated with MOG440F, scrambled MOG37-52 or ovalbumin are not capable of stimulating T-cell proliferation.

FIG. 6A illustrates nanoparticles coated with MOG37-52 or MOG44βF suppress the development of MOG-induced EAE. C57BL/6 mice were injected intravenously with 500 nm silica nanoparticles coated with MOG37-52, MOG44βF or scrambled MOG37-52 or bear nanoparticles on day −7 and day −1 (total 50 μg peptide per mouse). On day 0 mice were immunised with MOG35-55 in complete Freund's adjuvant to induce EAE.

FIG. 6B illustrates nanoparticles coated with MOG37-52 or MOG44βF suppress the development of MOG-induced EAE. Kaplan-Meier time-to-score-3 plot. *p<0.05, **p<0.01, ***p<0.001, ****p<0.0001. One-way ANOVA with Tukey's post test was used.

FIGS. 7A-C illustrate nanoparticles coated with MOG37-52 or MOG443F suppress the development of MOG-induced EAE. Clinical outcomes of EAE mice treated with nanoparticles. FIG. 7A shows mean day of disease onset, FIG. 7B shows mean maximum clinical score, and FIG. 7C shows cumulative disease score. *p<0.05, **p<0.01, ***p<0.001, ****p<0.0001. One-way ANOVA with Tukey's post test was used.

FIGS. 8A-C illustrate nanoparticles coated with MOG37-52 or MOG443F significantly 30 reduce inflammation (FIG. 8A), demyelination (FIG. 8B), and axonal damage (FIG. 8C) within the central nervous system of EAE mice. Spinal cord sections from EAE mice were stained with hematoxylin and eosin, Luxol fast blue or Bielschowsky silver stain and semi-quantitative analysis of inflammation, demyelination and axonal damage, respectively, was performed blind to treatment. Mice that received 500 nm silica nanoparticles coated 5 with MOG37-52 or MOG44βF displayed significantly reduced inflammation, demyelination and axonal damage compared to mice that received bear nanoparticles or nanoparticles coated with scrambled MOG37-52. **p<0.01, ***p<0.001, ****p<0.0001. One-way ANOVA with Tukey's post test was used.

FIG. 9A illustrates nanoparticles coated with MOG37-52 suppress the development of MOG-induced EAE. C57BL/6 mice were injected intravenously with 200 nm silica nanoparticles coated with MOG37-52, scrambled MOG37-52 or ovalbumin, or bear nanoparticles on day −7 and day −1 (total 50 μg peptide per mouse). On day 0 mice were immunised with MOG35-55 in complete Freund's adjuvant to induce EAE.

FIG. 9B illustrates nanoparticles coated with MOG37-52 suppress the development of MOG-induced EAE. Kaplan-Meier time-to-score-3 plot.

FIGS. 9C-E illustrate nanoparticles coated with MOG37-52 suppress the development of MOG-induced EAE. Clinical outcomes of EAE mice treated with nanoparticles. FIG. 9C shows mean day of disease onset, FIG. 9D shows mean maximum clinical score, and FIG. 9E shows cumulative disease score. *p<0.05, **p<0.01, ***p<0.001, ****p<0.0001. One-way ANOVA with Tukey's post test was used.

FIGS. 10A-C illustrate nanoparticles coated with MOG37-52 significantly reduce inflammation and axonal damage within the central nervous system of EAE mice. Spinal cord sections from EAE mice were stained with hematoxylin and eosin, Luxol fast blue or Bielschowsky silver stain and semi-quantitative analysis of (FIG. 10A) inflammation, (FIG. 10 B) demyelination and (FIG. 10C) axonal damage, respectively, was performed blind to treatment. Mice that received 200 nm silica nanoparticles coated with MOG37-52 displayed significantly reduced inflammation compared to mice that received bear nanoparticles, and significantly reduced axonal damage compared to mice that received bear nanoparticles or nanoparticles coated with scrambled MOG37-52 or ovalbumin. *p<0.05, ***p<0.001. One-way ANOVA with Tukey's post test was used.

FIG. 11A illustrates nanoparticles coated with MOG37-52 suppress the development of MOG-induced EAE. C57BL/6 mice were injected intravenously with 200 nm silica nanoparticles coated with MOG37-52, scrambled MOG37-52 or ovalbumin, or bear nanoparticles on day −7 and day −1 (total 50 μg peptide per mouse). On day 0 mice were immunised with recombinant mouse MOG in complete Freund's adjuvant to induce EAE.

FIG. 11B illustrates nanoparticles coated with MOG37-52 suppress the development of MOG-induced EAE. Kaplan-Meier time-to-score-3 plot.

FIG. 11C-E illustrate nanoparticles coated with MOG37-52 suppress the development of MOG-induced EAE. Clinical outcomes of EAE mice treated with nanoparticles. FIG. 1C shows mean day of disease onset, FIG. 11D shows mean maximum clinical score, and FIG. 11E shows cumulative disease score. *p<0.05, **p<0.01. One-way ANOVA with Tukey's post test was used.

FIG. 12 illustrates in vivo analysis of the IL-17A secretion profile of splenocytes from the vaccinated mice further revealed a significant decrease in the production of the pro-inflammatory cytokine.

### DETAILED DESCRIPTION

While the invention has been disclosed with reference to certain embodiments, it will be understood by those skilled in the art that various changes may be made and equivalents may be substituted without departing from the scope of the invention. In addition, many modifications may be made to adapt to a particular situation or material to the teachings of the invention without departing from its scope.

Throughout the specification and claims, the following terms take the meanings explicitly associated herein unless the context clearly dictates otherwise. The meaning of “a”, “an”, and “the” include plural references. The meaning of“in” includes “in” and “on.” Referring to the drawings, like numbers indicate like parts throughout the views. Additionally, a reference to the singular includes a reference to the plural unless otherwise stated or inconsistent with the disclosure herein.

The word “exemplary” is used herein to mean “serving as an example, instance, or illustration.” Any implementation described herein as “exemplary” is not necessarily to be construed as advantageous over other implementations.

Although the detailed description contains many specifics, these should not be construed as limiting the scope of the invention but merely as illustrating different examples and aspects of the invention. It should be appreciated that the scope of the invention includes other embodiments not discussed herein. Various other modifications, changes and variations which will be apparent to those skilled in the art may be made in the arrangement, operation and details of the system and method of the present invention disclosed herein without departing from the spirit and scope of the invention as described here.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

For treatment of demyelination disorders, e.g. multiple sclerosis, an advantage of embodiments of the present invention is that the peptides used herein in compositions of the invention or coupled to carrier particles have reduced encephalitogenic potential compared to other peptides derived from naked MOG autoantigens, such as MOG35-55. This is particularly advantageous for use in patients that are susceptible to induction or accelerating progression of multiple sclerosis. Further, lower doses of peptide and reduced number of doses are required to achieve a beneficial effect when a composition or carrier particle of the invention is used compared to naked peptide and adjuvant in various model systems.

Nanoparticles coupled with peptides designed to mimic immunodominant epitopes can directly target antigen-specific T cells without compromising the normal functioning of the immune system, and is advantageous over non-specific suppression or halting of the immune system which leaves subjects susceptible to severe side effects or opportunistic infections.

Substitution of an α-amino acid which binds to or interacts with a T cell receptor or an MHC molecule with the β-amino acid form retains the side chain and the native Cα-Cβ orientation but incorporates a methyl insertion extending the backbone of the molecule. Without being bound by any theory or mode of action, it is believed that the changes in the strength of the interactions between the T cell receptor and the MHC complex results in differential T cell signalling and effector functions, such as a reduction in inflammatory cytokines and increased numbers of T reg cells.

In some embodiments, the silica nanoparticles compared to PLGA and polystyrene, possess significant advantage of better immune response in terms of: a) enhanced uptake by mononuclear phagocytic system leading to accumulation in macrophages at liver, spleen and lymph nodes, b) enhanced surface area of nanoparticles of up to 20 fold higher compared to same size of solid nanoparticles, c) higher loading of peptide antigen, and d) biodegradable by macrophages within 1-2 weeks. All these unique features make silica suitable for delivery of immunosuppressive antigens to macrophages.

In some embodiments, the nano- or micro-carrier containing silica is conjugated to or loaded with a modified MOG 44βF antigen to preferentially accumulate the conjugates in the spleen and liver macrophages where the immunomodulatory activities take place. In some embodiments, intravenous injections of nano peptide conjugates suppress the disease at a very low dose of 25-50 μg compared to 800 μg in case of free peptide or 15-30 fold lower dose than the free peptide. In some embodiments, specific targeting of therapeutic peptides to spleen and liver macrophages has great advantage in inducing suppression of autoimmunity. In some embodiments, the engineered compositions provide magnetic resonance contrast and near-infrared fluorescence in vivo such that the biodistribution, particularly the liver/spleen uptake and clearance, can be imaged and quantified non-invasively. In some embodiments, the dosing is monitored in vivo. In some embodiments, the biodegradable carrier degrades away within 2-3 days following administration of the carrier particle or the composition in a subject.

In some embodiments, provided is a composition comprising a myelin basic protein (MBP) peptide coupled to a carrier particle, wherein the peptide comprises an amino 30 acid sequence having at least 60%, 70%, 75%, 80%, 81%, 82%, 83%, 84%, 85%, 86%, 87%, 88%, 89%, 90° 6, 91%, 92%, 93%, 94%, 95%, 96%, 97%, 98% or 99% sequence identity with SEQ ID NO: 8, wherein the amino acid residue at, or equivalent to: position 91 in SEQ ID NO: 8 is not an αLys: and/or position 96 in SEQ ID NO: 8 is not an αPro. In some embodiments, the amino acid residue is a non-conservative substitution relative to the amino acid that occurs in that position in SEQ ID NO: 8. In one embodiment, the amino acid is alanine or a β amino acid, or a β amino acid version of the amino acid that occurs at that position in SEQ ID NO: 8. In one embodiment, the amino acid at position 91 or 96 is either an α-alanine, β-phenylalanine or β-alanine.

In some embodiments, provided is a composition comprising a peptide having a myelin basic protein (MBP) peptide having a β amino acid at a position, or position equivalent to, 91 and/or 96 in SEQ ID NO: 8 (numbering wherein Val at the N-terminus is position 87 and Pro at the C-terminus is position 99), the peptide coupled to a carrier particle.

In other embodiments, provided is a composition comprising a proteolipid protein (PLP) peptide coupled to a carrier particle, wherein the peptide comprises an amino acid sequence having at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 9, wherein the amino acid residue at, or equivalent to: position 141 in SEQ ID NO: 9 is not an αLeu; position 144 in SEQ ID NO: 9 is not an αTrp: position 145 in SEQ ID NO: 9 is not an αLeu: position 147 in SEQ ID NO: 9 is not an αHis; and/or position 148 in SEQ ID NO: 9 is not an αPro. In some embodiments, the amino acid residue is a non-conservative substitution relative to the amino acid that occurs in that position in SEQ ID NO: 9. In one embodiment, the amino acid is alanine or a β amino acid, or a β amino acid version of the amino acid that occurs at that position in SEQ ID NO: 9. In one embodiment, the amino acid at position 141, 144, 145, 147 or 148 is an α-alanine, β-phenylalanine or β-alanine.

In some embodiments, provided is a composition comprising a peptide having a proteolipid protein (PLP) peptide having a β amino acid at a position, or position equivalent to, 141, 144, 145, 147, and/or 148 in SEQ ID NO: 9 (numbering wherein His at the N-terminus is position 139 and Phe at the C-terminus is position 151), the peptide coupled to a carrier particle.

In some embodiments, provided is a composition comprising a peptide having an amino acid sequence with 60% identity to SEQ ID NO: 1, the peptide coupled to a carrier particle. The present invention also provides a carrier particle conjugated to or encapsulated with a peptide having an amino acid sequence with 60% identity to SEQ ID NO: 1. In one embodiment, the peptide is less than 21 amino acids in length. In some embodiments, the peptide is 20, 19, 18, 17 or 16 amino acids in length. In some embodiments, the peptide comprises an amino acid sequence that is at least 60, 70, 80, 90, 91, 92, 93, 94, 95, 96, 97, 98, 99 or 100% identity to SEQ ID NO: 1.

In other embodiments, a peptide of the invention consists of or consisting essentially of an amino acid sequence that is at least 60, 70, 80, 90, 91, 92, 93, 94, 95, 96, 97, 98, 99 or 100% identical to SEQ ID NO: 1. In these embodiments, a peptide that includes an amino acid sequence of SEQ ID NO: 1 as well as additional amino acid residues would “consist essentially of” SEQ ID NO: 1 as long as it exhibits activity for stimulating proliferation of MOG-specific T cells, as may be determined in accordance with the assays described below. In some embodiments, the MOG-specific T cells proliferate in response to a peptide consisting of an amino acid sequence of 37 to 52 of SEQ ID NO: 1. Similarly, a peptide “consists essentially of” SEQ ID NO: 1 includes peptides where the length is shorter than the corresponding SEQ ID as long as it exhibits activity for stimulating proliferation of MOG-specific T cells, as may be determined in accordance with the assays described below. In one embodiment, the MOG-specific T cells proliferate in response to a peptide consisting of an amino acid sequence of 37 to 52 of SEQ ID NO: 1.

A peptide, or peptide antigen, of the invention may be isolated, purified, substantially purified, enriched, synthetic or recombinant.

In some embodiments, a pharmaceutical composition for treating or preventing an autoimmune disease comprising a composition or carrier particle as described herein and a pharmaceutically acceptable diluent, excipient or carrier. In one embodiment, the autoimmune disease is a demyelination disorder such as multiple sclerosis.

In one embodiment the subject is identified as being at risk of developing multiple sclerosis. For example, the subject may have a family history of multiple sclerosis or exhibit clinical signs of a precursor condition to, or early stage, multiple sclerosis, such as clinically isolated syndrome.

In one embodiment, provided herein is use of a composition in the manufacture of a medicament for the treatment or prevention of an autoimmune disease. In some embodiments, the autoimmune disease is a demyelination disorder such as multiple sclerosis.

In any embodiment of a method or use of the invention described herein, the composition or particle may be administered to the subject prior to an episode, during exacerbation or attack of symptoms of a demyelination disorder, such as multiple sclerosis.

In another embodiment of a method or use of the invention described herein, the step of in vitro screening the subject to be treated for T cells that respond (e.g. proliferate) to a composition or peptide is as described herein. In some embodiments, the T cells are obtained from the subject and then the proliferation of those T cells to a composition or peptide as described herein is performed using an assay as described herein.

A process for the preparation of a carrier particle of silica is provided as described herein, the process comprises a precursor, monomer solution for the carrier particle and a method of hydrolysing, condensing, precipitating, or aggregating the precursor, or monomer into particles, purification of the particles from the reactants and solvents, surface functionalizing the particles with carboxyl, amine or thiol groups and conjugating the functional groups with peptide antigen. A method of encapsulating the peptide with the carrier particle is provided by way of first reacting or complexing the carrier precursor, dissolved monomer with said peptide ligand in a common solvent and precipitating the complex or reaction mixture using a non-solvent, followed by washing and purification. The carrier particles are designed to provide magnetic resonance contrast property such that the biodistribution of the conjugates can be imaged non-invasively using MRI, thereby determining the dosing. The carrier particles are designed to provide fluorescent property such that the live in vivo biodistribution of the conjugates can be imaged by near infrared in vivo imaging. The carrier particles are designed to get preferentially accumulated in the macrophages of spleen and liver to deliver antigens for immune modulation such as inducing tolerance in the case of autoimmune disease and spleen macrophages down-regulate or suppress the auto-immune reaction related interleukin IL-17.

In another aspect, the concentration of the peptide and the method of surface conjugation on the carrier nano-, or micro-particle are optimized to evoke desired immune response by way of macrophage-phagocytosis in liver and spleen. In yet another aspect, the nanoparticles are biodegraded by the macrophages in vivo. In one embodiment, the nanoparticle facilitate immune-suppression of autoreactive T cells using a concentration of drug at least 15-30 times lower than free drug.

As used herein, the phrase “autoimmune disease” refers generally to those diseases characterized by the failure of one or more B- and/or T-cell populations, or gene products thereof, to distinguish between self and non-self antigenic determinants. Autoimmune diseases are often characterized by the infiltration of the target cells with inflammatory lymphoid cells, for example, mononuclear phagocytes, lymphocytes and plasma cells as well as secondary lymphoid follicles.

Examples of autoimmune diseases or disorders include, but are not limited to, inflammatory responses such as inflammatory skin diseases including psoriasis and dermatitis (e.g., atopic dermatitis); systemic scleroderma and sclerosis: responses associated with inflammatory bowel disease (such as Crohn's disease and ulcerative colitis): respiratory distress syndrome (including adult respiratory distress syndrome; ARDS): dermatitis; meningitis: encephalitis: uveitis: colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE): diabetes mellitus (e.g., Type I diabetes mellitus or insulin dependent diabetes mellitis); multiple sclerosis; Reynaud's syndrome; autoimmunethyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, inflammatory myopathies, interstitial lung disease, granulomatosis and vasculitis; pernicious anemia (Addison's disease); diseases involving leukocyte diapedesis; central nervous system (CNS) inflammatory disorder; multiple organ injury syndrome: hemolytic anemia (including, but not limited to cryoglobinemia or Coombs positive anemia); myasthenia gravis: antigen-antibody complex mediated diseases; anti-glomerular basement membrane disease; antiphospholipid syndrome: allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis: immune complex nephritis; IgA nephropathy: IgM polyneuropathies; immune thrombocytopenic purpura (ITP) or autoimmune thrombocytopenia, etc. In some embodiments, the autoimmune disease is T cell-mediated or T cell-dependent. In some embodiments, the autoimmune disease is B cell-mediated or B cell-dependent.

As used herein, a demyelination disorder is selected from the group consisting of multiple sclerosis, Acute Disseminated Encephalomyelitis, transverse myelitis, neuromyelitis optica, Guillain-Barre Syndrome (GBS), chronic inflammatory demyelinating polyneuropathy (CIDP), Anti-MAG Disease, multifocal motor neuropathy (MMN), Alzheimer's disease. In some embodiments, the demyelination disorder is selected from the group consisting of multiple sclerosis, Acute Disseminated Encephalomyelitis, transverse myelitis, neuromyelitis optica. In some embodiments, the demyelination disorder is multiple sclerosis. In some embodiments, the demyelination disorder is cell-mediated or dependent, i.e. T cell mediated or T cell dependent. In some embodiments, the autoimmune disease is B cell-mediated or B cell-dependent.

“Multiple sclerosis” refers to the chronic and often disabling disease of the central nervous system characterized by the progressive destruction of the myelin and is intended to encompass conditions which fall within the recognised diagnostic criteria described in Table 4 of Polman et al., (2011) Ann Neurol 69:292-302, the entire contents of which is incorporated herein by reference. Briefly, these diagnostic criteria rely upon one or a combination of clinical identification of at least one MS episode, and where there are multiple episodes their dissemination over time, and the pathophysiological identification of at least one causative lesion, and where there are multiple lesions their dissemination over space. Also amongst the range of clinical conditions intended to be encompassed by this definition are any one or more of relapse remitting multiple sclerosis, secondary progressive multiple sclerosis, relapsing progressive multiple sclerosis, chronic progressive multiple sclerosis and primary progressive multiple sclerosis.

Neurological signs associated with MS encompass a wide array of symptoms including limb weakness, compromised motor and cognitive function, sensory impairment, bladder disorders, sexual dysfunction, fatigue, ataxia, deafness and dementia. Despite variation in symptoms, the progression of several clinical courses has been classified. The majority of patients with MS follow a relapsing-remitting course in the early stages of the disease, characterised by increased severity of existing symptoms and the appearance of new symptoms, followed by variable periods of total or partial recovery. Relapsing-remitting MS (RRMS) may be inactive for several years between distinct attacks. However, most patients with RRMS ultimately enter a secondary chronic progressive phase, characterised by progressive disability and classified as secondary progressive MS (SPMS). This disease state may also involve relapses, thereby known as relapsing progressive MS (RPMS).

There are four internationally recognized forms of MS, namely, primary progressive multiple sclerosis (PPMS), relapsing-remitting multiple sclerosis (RRMS), secondary progressive multiple sclerosis (SPMS), and progressive relapsing multiple sclerosis (PRMS).

“Primary progressive multiple sclerosis” or “PPMS” is characterized by a gradual progression of the disease from its onset with no superimposed relapses and remissions at all. There may be periods of a levelling off of disease activity and there may be good and bad days or weeks. PPMS differs from RRMS and SPMS in that onset is typically in the late thirties or early forties, and initial disease activity is often in the spinal cord and not in the brain. PPMS often migrates into the brain, but is less likely to damage brain areas than RRMS or SPMS; for example, people with PPMS are less likely to develop cognitive problems. PPMS is the sub-type of MS that is least likely to show inflammatory (gadolinium enhancing) lesions on MRI scans. The Primary Progressive form of the disease affects between 10 and 15% of all people with multiple sclerosis. PPMS may be defined according to the criteria in Polman et al., (2011) Ann Neurol 69:292-302. The subject with PPMS treated herein is usually one with probable or definitive diagnosis of PPMS.

“Relapsing-remitting multiple sclerosis” or “RRMS” is characterized by relapses (also known as exacerbations) during which time new symptoms can appear and old ones resurface or worsen. The relapses are followed by periods of remission, during which time the person fully or partially recovers from the deficits acquired during the relapse. Relapses can last for days, weeks or months and recovery can be slow and gradual or almost instantaneous. The vast majority of people presenting with MS are first diagnosed with RRMS. This is typically when they are in their twenties or thirties, though diagnoses much earlier or later are known. Two to three times as many women as men present with this sub-type of MS. During relapses, myelin, a protective insulating sheath around the nerve fibers (neurons) in the white matter regions of the central nervous system (CNS), may be damaged in an inflammatory response by the body's own immune system. This causes a wide variety of neurological symptoms that vary considerably depending on which areas of the CNS are damaged. Immediately after a relapse, the inflammatory response dies down and a special type of glial cell in the CNS (called an oligodendrocyte) sponsors remyelination—a process whereby the myelin sheath around the axon may be repaired. It is this remyelination that may be responsible for the remission. Approximately 50% of patients with RRMS convert to SPMS within 10 years of disease onset. After 30 years, this figure rises to 90%. At any one time, the relapsing-remitting form of the disease accounts around 55% of all people with MS.

“Secondary progressive multiple sclerosis” or “SPMS” is characterized by a steady progression of clinical neurological damage with or without superimposed relapses and minor remissions and plateau. People who develop SPMS will have previously experienced a period of RRMS, which may have lasted anything from two to forty years or more. Any superimposed relapses and remissions there are, tend to tail off over time. From the onset of the secondary progressive phase of the disease, disability starts advancing much quicker than it did during RRMS though the progress can still be quite slow in some individuals. After 10 years, 50% of people with RRMS will have developed SPMS. By 25 to 30 years, that figure will have risen to 90%. SPMS tends to be associated with lower levels of inflammatory lesion formation than in RRMS but the total burden of disease continues to progress. At any one time, SPMS accounts around 30% of all people with multiple sclerosis.

“Progressive relapsing multiple sclerosis” refers to “PRMS” is characterized by a steady progression of clinical neurological damage with superimposed relapses and remissions. There is significant recovery immediately following a relapse but between relapses there is a gradual worsening of symptoms. PRMS affects around 5% of all people with multiple sclerosis. Some neurologists believe PRMS is a variant of PPMS.

As used herein the term “ameliorating multiple sclerosis” refers to any one or more of preventing, delaying, slowing or reversing the progression of the pathology and/or one or more symptoms of multiple sclerosis or preventing or delaying the establishment of multiple sclerosis in a human or non-human animal subject. Thus, in certain embodiments ameliorating multiple sclerosis is intended to encompass interfering with the progression of multiple sclerosis in a subject diagnosed or suspected as having multiple sclerosis. In certain embodiments ameliorating multiple sclerosis is intended to encompass preventing, interfering with or delaying the relapse of multiple sclerosis in a subject who has previously suffered from multiple sclerosis but who is currently in remission. By the terms “preventing” or “prevention”, it is intended that the nanoparticles, compositions and/or methods eliminate or reduce the incidence or onset of the disorder, as compared to that which would occur in the absence of the measure taken. In other words, the present methods slow, delay, control, or decrease the likelihood or probability of the disorder in the subject, as compared to that which would occur in the absence of the measure taken.

A “subject” herein may be a human subject. Although the invention finds application in humans, the invention is also useful for veterinary purposes. The invention is useful for domestic or farm animals such as cattle, sheep, horses and poultry; for companion animals such as cats and dogs; and for zoo animals.

Generally, the human subject is eligible for treatment for an autoimmune disease or demyelination disorder as described herein such as multiple sclerosis. For some purposes herein, such eligible subject is one who is experiencing, has experienced, or is likely to experience, one or more signs, symptoms or other indicators of multiple sclerosis; has been diagnosed with multiple sclerosis, whether, for example, newly diagnosed (with “new onset” MS), previously diagnosed with a new relapse or exacerbation, previously diagnosed and in remission, or is at risk for developing multiple sclerosis. One suffering from or at risk for suffering from multiple sclerosis may optionally be identified as one who has been screened for elevated levels of CD20-positive B cells in serum, cerebrospinal fluid (CSF) and/or MS lesion(s) and/or is screened for using an assay to detect autoantibodies, assessed qualitatively, or also quantitatively. Exemplary such autoantibodies associated with multiple sclerosis include anti-myelin basic protein (MBP), anti-myelin oligodendrocytic glycoprotein (MOG), anti-ganglioside and/or anti-neurofilament antibodies. Such autoantibodies may be detected in the subject's serum, cerebrospinal fluid (CSF) and/or MS lesion. By “elevated” autoantibody or B cell level(s) herein is meant level(s) of such autoantibodies or B cells which significantly exceed the level(s) in an individual without MS.

The words ‘treat’ or ‘treatment’ refer to treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects and the progression of a disease or disorder. The success or otherwise of treatment may be monitored by, amongst other things, clinical or biochemical examination.

The response of a subject with or at risk of MS to the administration of a composition or particle as described herein may be monitored by monitoring of the clinical condition of the subject, including but not limited to monitoring of the period of remission and time to relapse of MS, monitoring of the size and distribution of MS lesions by standard methods such MRI, and by monitoring the number and distribution of T reg cells and/or autoantigen specific effector T cells in secondary lymphoid organs.

Biochemical analysis of a subject receiving a therapy as described herein may show a reduction in inflammation, demyelination and axonal damage within the central nervous system.

Further, treatment of MS may refer to a decrease or reduction in severity of MS symptoms, an increase in frequency and duration of MS symptom-free periods, reduction in the progression of clinical stages, reduce the frequency of relapses, or a prevention of impairment or disability due to MS affliction. Clinical tests for measuring the efficacy of combination therapies for treating chronic progression measure the deterioration of physical symptoms associated with MS including, for example, relapse rate: vision loss: sensory loss: gait disorders, such as axial instability, hyperreflexia, loss of dexterity, and spasticity; bladder dysfunction, depression: and cognitive impairment.

Other clinical tests for measuring the efficacy of combination therapies for “treating chronic progression” include the expanded disability status scale (“EDSS”), the MS functional composite (“MSFC”), as well as MTR, NAA/Cr. Biochemical tests for measuring the efficacy of combination therapies for treating chronic progression of MS may measure levels of myelin, integrity of the blood-brain barrier, perivascular infiltration of mononuclear cells, immunologic abnormalities, gliotic scar formation and astrocyte proliferation, metalloproteinase production, and impaired conduction velocity.

Accordingly, any clinical or biochemical assay that monitors any of the foregoing may be used to determine whether a particular treatment is efficacious for treating chronic progression of MS in an individual following treatment of the individual with the methods of the present invention.

As used herein, the term “reduction in severity” refers to an arrest, decrease, or reversal in signs, symptoms, physiological indicators, biochemical markers or metabolic indicators associated with MS. Symptoms of MS include, for example, neurological impairment and neuroinflammation. Physiological indicators of MS include, for example, demyelination of nerve fibers. Biochemical markers indicative of MS include, for example, myelin and gamma globulin. Demyelination and remyelination of nerve fibers may be detected by clinical methods known to those of skill in the art. For example, evoked potentials may be used to measure the speed with which nerve impulses travel along nerve fibers throughout the nervous system. Additionally, computer-assisted tomography (CT) may be used to scan the nervous system to detect nerve fiber demyelination or remyelination. Magnetic resonance imaging (MRI) also may be used to scan the nervous system to detect nerve fiber demyelination or remyelination without the use of x-rays. Such MRI-based tests for measuring the efficacy of combination therapies for treating chronic progression, measure, for example, enhancing lesions, T1 black holes and T2 lesion load. Lumbar punctures or spinal taps may be used to withdraw cerebrospinal fluid, which subsequently may be tested for levels of biochemical markers, such as, for example, myelin or gamma globulin. Accordingly, any of the foregoing may be used to detect whether the severity of MS symptoms has been reduced in a patient following treatment of the individual using the methods of the present invention. Certain of the tests for monitoring the efficacy of treating chronic progression of MS may be used interchangeably with tests for monitoring the efficacy of reducing the severity of MS symptoms as is known to those of skill in the art.

As used herein, a subject “at risk” of developing a disease or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

Any suitable antigens may find use within the scope of the present invention. In some embodiments, the inducing antigen (i.e. the antigen in a composition or coupled to a particle of the invention) contributes to the specificity of the tolerogenic response that is induced. The inducing antigen may or may not be the same as the target antigen, which is the antigen present or to be placed in the subject being treated which is a target for the unwanted immunological response, and for which tolerance is desired.

The inducing antigen which may be identical with or immunologically related to the target antigen, however the inducing antigen has at least one β amino acid which is not present in the target antigen. There is also the additional option of using an antigen which is a bystander for the target. This is an antigen which may not be immunologically related to the target antigen, but is preferentially expressed in a tissue where the target antigen is expressed. Again, the inducing antigen has at least one β amino acid which is not present in the target antigen. In certain embodiments of this invention, the inducing antigen is not in the same form as expressed in the individual being treated, but is a fragment or derivative thereof. Inducing antigens of this invention include peptides based on a molecule of the appropriate specificity but adapted by fragmentation, residue substitution, labeling, conjugation, and/or fusion with peptides having other functional properties.

In any embodiment described herein, the antigen comprises, or comprises at least a portion of an autoimmune antigen, an antigen expressed on a tissue to be transplanted into a subject, or an allergen. In some embodiments, the antigen is one or more selected from: myelin basic protein (MBP), acetylcholine receptor, endogenous antigen, myelin oligodendrocyte glycoprotein (MOG), myelin-associated glycoprotein, β-amyloid, pancreatic beta-cell antigen, insulin, proinsulin, islet-specific glucose-6-phophatase catalytic subunit-related protein (IGRP), glutamic acid decarboxylase (GAD), collagen type 11, human cartilage gp39, fp130-RAPS, proteolipid protein (PLP), fibrillarin, small nucleolar protein, thyroid stimulating factor receptor, histones, glycoprotein gp70, pyruvate dehydrogenase dehydrolipoamide acetyltransferase (PCD-E2), hair follicle antigen, aqua porin 4, Desmoglein 1, Desmoglein 3, nicotinic acetylcholine receptor, A-gliaden, human tropomyosin isoform 5, Bahia grass pollen (BaGP), peach allergen Pru p 3, alpha s1-Casein milk allergen, Apig1 celery allergen, Bere1 Brazil nut allergen, β-Lactoglobulin Milk allergen, Bovine serum albumin, Cor a 1.04 hazelnut allergen, ovalbumin egg allergen, Advate, antihemophilic factor, Kogenate, Eloctate, recombinant factor VIII Fc fusion protein, Refacto, Novo Vila, recombinant factor VII, eptacog alfa, Helixate, Monanine, Coagulation Factor IX, Wilate, Ceredase, Alglucerase, Cerezyme, Imiglucerase, Elelso, taliglucerase alfa, Fabrazyme, Agalsidase beta, Aldurazyme, Iduronidase, Myozyme, Acid-glucosidase, Elaprase, iduronate-2-sulfatase, Naglazyme arylsufatase B, or N-acetylgalactosamin e-4-sulfatase, proteinaceous therapies used in enzyme or 15 coagluation factor replacement such as myozyme, alglucerase, imiglucerase, taliglucerase, agalsidase beta, 1-iduronidase, acid glucosidase, Iduronate-2-sulfatase, N-acetylgalactosamnie-4-sulfatase, antihemophilic factor, factor VII, eptacogalfa, factor IX, miglustat, romiplostim, epoetin alpha, protein C, laronidase, lumizyme or Factor VIII. Further, any antigen comprising one or more epitopes associated with an autoimmune disease described herein is also contemplated.

Any one of the antigens above may have a β amino acid introduced by any means known to those skilled in the art, including during peptide synthesis or during recombinant production. One or more β amino acids may be introduced at position where the homologous α-amino acid is known to, or determined to, interact, bind to, or make contact with a T cell receptor or MHC molecule, such as, a class II MHC molecule. Assays to determine whether an α-amino acid interacts, binds or makes contact with T cell receptor or MHC molecule include synthesis of a series of overlapping and/or truncated peptides and/or peptides containing single substitutions such as alanine, lysine or arginine substitutions and; testing of their ability to induce the proliferation of T cells or T cell hybridomas reactive against the native protein or peptide in vitro, whereby T cell proliferation in response to the overlapping and/or truncated peptides would indicate that the modified peptide contains amino acid residues that interact with the T cell receptor (Shetty et al., (2014) Neurol Neuroimmunol Neuroinflammation 1:e22: Kuchroo et al., (1994) J Immunol 153:3326-3336): testing of their ability to induce the proliferation of T cells or T cell hybridomas reactive against the native protein or peptide in vitro, whereby a lack of, or reduction in, proliferation to the substituted peptide would indicate that the substituted amino acid interacts with the T cell receptor (Petersen et al., (2004) Eur J Immunol 34:165-173; Kuchroo et al., (1994) J Immunol 153:3326-3336); or pre-incubation of antigen presenting cells with the overlapping, truncated or substituted, and subsequent testing of their ability to compete with the induce the proliferation of T cells or T cell hybridomas reactive against the native protein or peptide in vitro in the presence of sub-optimal concentrations of the native protein or peptide, whereby no reduction in proliferation would indicate that the modified peptide contains amino acid residues contribute to MHC binding (Kuchroo et al., (1994) J Immunol 153:3326-333).

A beta (β) amino acid is an amino acid in which the amino group is bonded to the β carbon rather than the α carbon as in the 20 standard biological amino acids. Glycine lacks a β carbon such that β-glycine is not possible. Reference to a β amino acid is therefore to any amino acid other than glycine. A beta amino acid may be either C2 or C3, where the organic residue is next to the carbonyl group or amino group in a peptide, respectively. A β amino acid in a peptide maintains the Cα-Cβ orientation but incorporates a methyl insertion extending the backbone of the molecule. Unlike conservative substitutions of naturally occurring α (alpha) amino acids insertion of a homologous β amino acid allows for the constitutive side chain to be maintained.

In some embodiments, a beta (β) amino acid containing peptide as described herein has reduced encephalitogenic potential compared to the native peptide containing the homologous α-amino acid. “Myelin Oligodendrocyte Glycoprotein” (MOG) is a glycoprotein believed to be important in the process of myelinization of nerves in the central nervous system (CNS). In humans this protein is encoded by the MOG gene. It is speculated to serve as a necessary “adhesion molecule” to provide structural integrity to the myelin sheath and is known to develop late on the oligodendrocyte. GenBank accession numbers of exemplary mRNA and protein sequences of the MOG gene include NM_001008228.2 and NP_001008229.1, respectively. The sequence associated with each of these GenBank accession numbers, and all other sequence of isoforms, splice variants, paralogs or orthologs is incorporated by reference for all purposes.

In some embodiments, a MOG peptide useful in the present invention has reduced encephalitogenic potential compared to MOG 35-55, which may be determined by methods known in the art and as described herein. In some embodiments, the peptide cannot induce a clinically observable experimental autoimmune encephalomyelitis (EAE) in an animal model, such as a mouse, in the absence of adjuvant.

“Myelin basic protein” (MBP) and “proteolipid protein” (PLP) are also believed to be important in the process of myelinization of nerves in the central nervous system (CNS). GenBank accession numbers of an exemplary PLP mRNA sequence is NM_000533.4 and PLP amino acid sequence is NP_000524.3. GenBank accession numbers of an exemplary MBP mRNA sequence is NM_00102508.1 and MBP amino acid sequence is NP_001020252.1 The sequence associated with each of these GenBank accession numbers, and all other sequence of isoforms, splice variants, paralogs or orthologs is incorporated by reference for all purposes.

As used herein an amino acid residue at the position equivalent to position in SEQ ID NO: 1, 7, 8 or 9, or other sequence described herein, can be determined by any means known to a person skilled in the art. For example, an alignment of one or more sequences with an amino acid sequence of SEQ ID NO: 1, 7, 8 or 9, would allow a person skilled in the art to determine the amino acid at the position equivalent to position in SEQ ID NO: 1, 7, 8 or 9. A person skilled in the art can compare the three dimensional structure of a peptide with the three dimensional structure of a peptide having the amino acid sequence of SEQ ID NO: 1, 7, 8 or 9 and determine the amino acid residue that is at an equivalent position to that in SEQ ID NO: 1, 7, 8 or 9.

Myelin oligodendrocyte glycoprotein (MOG) derived peptides coupled to 30 nanoparticles and useful in the present invention include but are not limited to: MOG37-52 (human): VGWYRPPFSRVVHLYR (SEQ ID NO: 1: as used herein numbering with reference to SEQ ID NO: 1 is Val at the N-terminus is position 37 and Arg at the C-terminus is position 52): MOG44βF (human): VGWYRPPβFSRVVHLYR (SEQ ID NO: 2); MOG35-55 (human): MEVGWYRPPFSRVVHLYRNGK (SEQ ID NO: 3); MOG37-52 (mouse): VGWYRSPFSRVVHLYR (SEQ ID NO: 4); MOG443F (mouse): VGWYRSPβFSRVVHLYR (SEQ ID NO: 5); MOG35-55 (mouse): MEVGWYRSPFSRVVHLYRNGK (SEQ ID NO: 6): MOG119-132 (human): FYWVNPGVLTLIAL (SEQ ID NO: 7).

A full length amino acid sequence of human MOG is:

(SEQ ID NO: 10)
MASLSRPSLPSCLCSFLLLLLLQVSSSYAGQFRVIGPRHPIRALVGDEVE
LPCRISPGKNATGMEVGWYRPPFSRVVHLYRNGKDQDGDQAPEYRGRTEL
LKDAIGEGKVTLRIRNVRFSDEGGFTCFFRDHSYQEEAAMELKVEDPFYW
VSPGVLVLLAVLPVLLLQITVGLIFLCLQYRLRGKLRAEIENLHRTFESF
GVLGPQVKEPKKTGQFLEELRNPF.

Other peptide antigens useful for coupling to nanoparticles and useful in the present invention include but are not limited to: myelin basic protein including the peptide: MBP87-99 VHFFKNIVTPRTP (SEQ ID NO: 8) and proteolipid protein (PLP) including the peptide: PLP139-151 HSLGKWLGHPDKF (SEQ ID NO: 9).

“Percent (%) amino acid sequence identity” or “percent (%) identical” with respect to a peptide sequence, i.e. a peptide defined herein, is defined as the percentage of amino acid residues in a candidate sequence that are identical with the amino acid residues in the specific peptide of the invention, after aligning the sequences and introducing gaps, if necessary, to achieve the maximum percent sequence identity, and not considering any conservative substitutions as part of the sequence identity. Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A non-limiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting examples of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif. USA). When utilizing the ALIGN program for comparing amino acid sequences, a PAM 120 weight residue table, a gap length penalty of 12, and a gap penalty of 4 can be used.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid). Conservative amino acid substitution tables providing functionally similar amino acids are well known to one of ordinary skill in the art. The following six groups are examples of amino acids that may be considered to be conservative substitutions for one another: Alanine (A), Serine (S), Threonine (T); Aspartic acid (D), Glutamic acid (E); Asparagine (N), Glutamine (Q); Arginine (R), Lysine (K); Isoleucine (I), Leucine (L), Methionine (M), Valine (V); and Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogs (synthetic amino acids) well known in the art. A peptidomimetic of the naturally occurring amino acid is well documented in the literature known to the skilled person and non-natural or unnatural amino acids are described further below. When affecting conservative substitutions the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH2)5-COOH]—CO— for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue: (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Alterations of the native amino acid sequence to produce mutant peptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42: 133 (1986); Bauer et al., Gene 37: 73 (1985); Craik, Biotechniques, 12-19 (January 1995): and U.S. Pat. Nos. 4,518,584 and 4,737,462. A preferred means for introducing mutations is the QuikChange Site-Directed Mutagenesis Kit (Stratagene. LaJolla, Calif.).

Any appropriate expression vector (e.g., as described in Pouwels et al., Cloning Vectors: A Laboratory Manual (Elsevier, N.Y.: 1985)) and corresponding suitable host can be employed for production of recombinant peptides. Expression hosts include, but are not limited to, bacterial species within the genera Escherichia. Bacillus, Pseudomonas, Salmonella, mammalian or insect host cell systems including baculovirus systems (e.g., as described by Luckow et al., Bio/Technology 6: 47 (1988)), and established cell lines such as the COS-7, C127, 3T3, CHO, HeLa, and BHK cell lines, and the like. The skilled person is aware that the choice of expression host has ramifications for the type of peptide produced. For instance, the glycosylation of peptides produced in yeast or mammalian cells (e.g., COS-7 cells) will differ from that of peptides produced in bacterial cells, such as Escherichia coli.

Alternately, a peptide as described herein can be synthesized using standard peptide synthesizing techniques well-known to those of ordinary skill in the art (e.g., as summarized in Bodanszky, Principles of Peptide Synthesis (Springer-Verlag, Heidelberg: 1984)). In particular, the peptide can be synthesized using the procedure of solid-phase synthesis (see, e.g., Merrifield, J. Am. Chem. Soc. 85: 2149-54 (1963): Barany et al., Int. J. Peptide Protein Res. 30: 705-739 (1987); and U.S. Pat. No. 5,424,398). If desired, this can be done using an automated peptide synthesizer. Removal of the t-butyloxycarbonyl (t-BOC) or 9-fluorenylmethyloxycarbonyl (Fmoc) amino acid blocking groups and separation of the peptide from the resin can be accomplished by, for example, acid treatment at reduced temperature. The peptide-containing mixture can then be extracted, for instance, with dimethyl ether, to remove non-peptidic organic compounds, and the synthesized peptide can be extracted from the resin powder (e.g., with about 25% w/v acetic acid). Following the synthesis of the peptide, further purification (e.g., using high performance liquid chromatography (HPLC)) optionally can be done in order to eliminate any incomplete peptides or free amino acids. Amino acid and/or HPLC analysis can be performed on the synthesized peptide to validate its identity. For other applications according to the invention, it may be preferable to produce the peptide as part of a larger fusion protein, such as by the methods described herein or other genetic means, or as part of a larger conjugate, such as through physical or chemical conjugation, as known to those of ordinary skill in the art and described herein.

A peptide as described herein may also be modified by, conjugated or fused to another moiety or carrier peptide to facilitate purification, or increasing the in vivo half-life of the peptides, or for use in immunoassays using methods known in the art. For example, a peptide of the invention may be modified by glycosylation, acetylation, pegylation, phosphorylation, amidation, derivatization by known protecting/blocking groups, proteolytic cleavage, linkage to a cellular ligand or other protein, etc.

A “peptidomimetic” is a synthetic chemical compound that has substantially the same structure and/or functional characteristics of a peptide as described herein, the latter being described further herein. Typically, a peptidomimetic has the same or similar structure as a peptide as described herein, for example the same or similar sequence of SEQ ID NO: 1 or fragment thereof. A peptidomimetic generally contains at least one residue that is not naturally synthesised. Non-natural components of peptidomimetic compounds may be according to one or more of: a) residue linkage groups other than the natural amide bond (‘peptide bond’) linkages: b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

Peptidomimetics can be synthesized using a variety of procedures and methodologies described in the scientific and patent literatures, e.g., Organic Syntheses Collective Volumes, Gilman et al. (Eds) John Wiley & Sons, Inc., NY, al-Obeidi (1998) Mol. Biotechnol. 9:205-223; Hruby (1997) Curr. Opin. Chem. Biol. 1:114-119; Ostergaard (1997) Mol. Divers. 3:17-27; Ostresh (1996) Methods Enzymot. 267:220-234

A composition or particle of the invention may induce antigen-specific tolerance in a subject after administration (i.e. tolerate or tolerize the subject). In some embodiments, the administration of the particles to a subject results in activation induced death of effector T cells; anergy of effector T cells; apoptosis of effector T cells: conversion of effector T cells to regulatory T cells; induction and expansion of both antigen specific and non-specific regulatory T cells: isolation of effector T cells in the lymph nodes and spleen inhibiting their ability to traffic to peripheral sites and cause inflammation; and/or down regulation of T cell dependent antibody production.

As used herein, the term “anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the API sequence that can be found within the enhancer.

As used herein, ‘particle’, ‘nanosphere’ or ‘nanoparticle’ ‘(NP)’ refers to small discrete particle that is administered singularly or plurally to a subject, cell specimen or tissue specimen as appropriate. In some embodiments, the particles are a sphere, substantially spherical in shape, or sphere-like entity, bead, or liposome. In certain embodiments, the nanoparticle is not a liposome or viral particle. The particle may be solid or porous. The term “substantially spherical,” as used herein, means that the shape of the particles does not deviate from a sphere by more than about 10%.

In some embodiments, an antigenic peptide molecule is coupled to the carrier particle by a conjugate molecule and/or linker group. In some embodiments, coupling of the antigenic peptide to the carrier comprises one or more covalent and/or non-covalent interactions. The particle may be biocompatible and/or bioabsorbable. Particles can have a structure of variable dimension.

Particles typically consist of a substantially spherical core and optionally one or more layers. The core may vary in size and composition. In addition to the core, the particles may have one or more layers to provide functionalities appropriate for the applications of interest. The thicknesses of layers, if present, may vary depending on the needs of the specific applications. For example, layers may impart useful optical properties.

Layers may also impart chemical or biological functionalities, referred to herein as chemically active or biologically active layers, and for these functionalities the layer or layers may typically range in thickness from about 0.001 micrometers (1 nanometer) to about 10 micrometers or more (depending on the desired nanoparticle diameter), these layers typically being applied on the outer surface of the particle.

The compositions of the core and layers may vary. Suitable materials for the particles or the core include, but are not limited to polymers, ceramics, glasses, minerals, and the like. Examples include, but are not limited to, standard and specialty glasses, silica, polystyrene, polyester, polycarbonate, acrylic polymers, polyacrylamide, polyacrylonitrile, polyamide, polylactic acid, polyglycolic acid, polycaprolactone, polyglutamic acid, polyglutamic acid, fluoropolymers, silicone, celluloses, silicon, metals (e.g., iron, gold, silver), minerals (e.g., ruby), nanoparticles (e.g., gold nanoparticles, colloidal particles, metal oxides, metal sulfides, metal selenides, and magnetic materials such as iron oxide), and composites thereof. The core could be of homogeneous composition, or a composite of two or more classes of material depending on the properties desired. In some embodiments, the composition of the particle is predominantly silica with or without atomic scale porosity. Exemplary methods for the production of silica nanoparticles are described in Stober et al. (1968). Journal of Colloid and Interface Science. 26, 62-69: Ibrahim et al. (2010) Journal of American Science 2010:6(11): 985:989; Tadanaga et al. (2013) J Sol-Gel Sci Technol. 68:341-345: and Nandy et al. (2014) J Sol-Gel Sci Technol. 72:49-55 the entire contents of each are incorporated herein by reference.

As previously stated, the particle may, in addition to the core, include one or more layers. The nanoparticle may include a layer consisting of a biodegradable sugar or other polymers. Examples of biodegradable layers include but are not limited to dextran: poly(ethylene glycol): poly(ethylene oxide): mannitol; poly(esters) based on polylactide (PLA), polyglycolide (PGA), polycaprolactone (PCL); poly(hydroxalkanoate)s of the PHB-PHV class; and other modified poly(saccharides) such as starch, cellulose and chitosan. Additionally, the particle may include a layer with suitable surfaces for attaching chemical functionalities for chemical binding or coupling sites.

Layers can be produced on the particles in a variety of ways known to those skilled in the art. Examples include sol-gel chemistry techniques such as described in Iler, Chemistry of Silica, John Wiley & Sons, 1979; Brinker and Scherer, Sol-gel Science, Academic Press, (1990). Additional approaches to producing layers on nanoparticles include surface chemistry and encapsulation techniques such as described in Partch and Brown. J. Adhesion, 67:259-276, 1998; Pekarek et al., Nature, 367:258, (1994); Hanprasopwattana, Langmuir, 12:3173-3179, (1996); Davies. Advanced Materials, 10:1264-1270, (1998); and references therein. Vapor deposition techniques may also be used; see for example Golman and Shinohara, Trends Chem. Engin., 6:1-6, (2000); and U.S. Pat. No. 6,387,498. Still other approaches include layer-by-layer self-assembly techniques such as described in Sukhorukov et al., Polymers Adv. Tech., 9(10-11):759-767, (1998); Caruso et al., Macromolecules, 32(7):2317-2328, (1998); Caruso et al., J. Amer. Chem. Soc., 121(25):6039-6046, (1999): U.S. Pat. No. 6,103,379 and references cited therein.

Preferred polymers for such preparations are natural or synthetic copolymers or polymers selected from the group consisting of gelatin agar, starch, arabinogalactan, albumin, collagen, polyglycolic acid, polylactic acid, glycolide-L(−) lactide poly(episilon-caprolactone, poly(epsilon-caprolactone-CO-lactic acid), poly(epsilon-caprolactone-CO-glycolic acid), poly((3-hydroxy butyric acid), poly(ethylene oxide), polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate).

Particularly preferred polymers are polyesters, such as polyglycolic acid, polylactic acid, glycolide-L(−) lactide poly(episilon-caprolactone, poly(epsilon-caprolactone-CO-lactic acid), and poly(epsilon-caprolactone-CO-glycolic acid. Solvents useful for dissolving the polymer include: water, hexafluoroisopropanol, methylenechloride, tetrahydrofuran, hexane, benzene, or hexafluoroacetone sesquihydrate.

It is not necessary that each particle be uniform in size, although the particles must generally be of a size sufficient to target the lymphoid organs and be taken up by antigen presenting cells such as macrophages. In some embodiments, the particles are microscopic or nanoscopic in size, in order to enhance solubility, avoid possible complications caused by aggregation in vivo and to facilitate pinocytosis. Particle size can be a factor for uptake from the interstitial space into areas of lymphocyte maturation. A particle having a diameter of from about 0.01 μm to about 10 μm is capable of triggering phagocytosis. Thus in one embodiment, the particle has a diameter within these limits.

In another embodiment, the particle has a diameter of about 0.3 μm to about 5 μm. In still another embodiment, the particle has a diameter of about 0.5 μm to about 3 μm. In a further embodiment the particle has a size of about 0.1 μm, or about 0.2 μm or about 0.3 μm or about 0.4 μm or about 0.5 μm or about 1.0 μm or about 1.5 μm or about 2.0 μm or about 2.5 μm or about 3.0 μm or about 3.5 μm or about 4.0 μm or about 4.5 μm or about 5.0 μm. In a particular embodiment the particle has a size of about 0.5 μm. In other embodiments, the nanoparticle is, or less than, about 500 nm, about 400 nm, about 300 nm, about 200 nm, about 100 nm, or about 50 nm in diameter. In further embodiments, the nanoparticle is from about 1 nm to about 10 nm, 15 nm, 20 nm, 25 nm, 30 nm, 40 nm, 50 nm, 75 nm, or 100 nm in diameter. In specific embodiments, the nanoparticle is from about 1 nm to about 100 nm, about 1 nm to about 50 nm, about 1 nm to about 20 nm, or about 5 nm to about 20 nm. In some embodiments, the overall weights of the particles are less than about 10,000 kDa, less than about 5,000 kDa. or less than about 1,000 kDa, 500 kDa, 400 kDa, 300 kDa, 200 kDa, 100 kDa, 50 kDa, 20 kDa, 10 kDa. The particles in a composition need not be of uniform diameter. By way of example, a pharmaceutical formulation may contain a plurality of particles, some of which are about 0.5 μm, while others are about 1.0 μm. Any mixture of particle sizes within these given ranges will be useful.

Pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton. Pa., 1980) discloses various carriers used in formulating pharmaceuticalcompositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose: starches such as corn starchand potato starch; cellulose and its derivatives such as sodium carboxymethyl cellulose, ethyl cellulose and cellulose acetate: powdered tragacanth; malt; gelatine; talc; excipients such as cocoa butter and suppository waxes: oils such as peanut oil, cottonseed oil: safflower oil, sesame oil; olive oil; corn oil and soybean oil; glycols; such as propylene glycol: esters such as ethyl oleate and ethyl laurate; agar: buffering agents such as magnesium hydroxide and aluminium hydroxide; alginic acid; pyrogenfree water: isotonic saline; Ringer's solution: ethyl alcohol, and phosphate buffer solutions, as well as other non-toxic compatible lubricants such as sodium lauryl sulfate and magnesium stearate, as well as colouring agents, releasing agents, coating agents, sweetening, flavouring and perfuming agents, preservatives and antioxidants can also be present in the composition, according to the judgment of the formulator.

Various dosage units are each provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain 30 aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

Compositions or particles of the invention may be formulated for local or topical administration, such as for topical application to the skin. Formulations for topical administration typically comprise a topical vehicle combined with active agent(s), with or without additional optional components.

Suitable topical vehicles and additional components are well known in the art, and it will be apparent that the choice of a vehicle will depend on the particular physical form and mode of delivery. Topical vehicles include organic solvents such as alcohols (for example, ethanol, iso-propyl alcohol or glycerine), glycols such as butylene, isoprene or propylene glycol, aliphatic alcohols such as lanolin, mixtures of water and organic solvents and mixtures of organic solvents such as alcohol and glycerine, lipid-based materials such as fatty acids, acylglycerols including oils such as mineral oil, and fats of natural or synthetic origin, phosphoglycerides, sphingolipids and waxes, protein-based materials such as collagen and gelatine, silicone-based materials (both nonvolatile and volatile), and hydrocarbon-based materials such as microsponges and polymer matrices.

A composition may further include one or more components adapted to improve the stability or effectiveness of the applied formulation, such as stabilizing agents, suspending agents, emulsifying agents, viscosity adjusters, gelling agents, preservatives, antioxidants, skin penetration enhancers, moisturizers and sustained release materials. Examples of such components are described in Martindale—The Extra Pharmacopoeia (Pharmaceutical Press, London 1993) and Martin (ed.), Remington's Pharmaceutical Sciences. Formulations may comprise microcapsules, such as hydroxymethylcellulose or gelatine-microcapsules, liposomes, albumin microspheres, microemulsions, nanoparticles or nanocapsules.

A topical formulation may be prepared in a variety of physical forms including, for example, solids, pastes, creams, foams, lotions, gels, powders, aqueous liquids, emulsions, sprays and skin patches. The physical appearance and viscosity of such forms can be governed by the presence and amount of emulsifier(s) and viscosity adjuster(s) present in the formulation. Solids are generally firm and non-pourable and commonly are formulated as bars or sticks, or in particulate form. Solids can be opaque or transparent, and optionally can contain solvents, emulsifiers, moisturizers, emollients, fragrances, dyes/colorants, preservatives and other active ingredients that increase or enhance the efficacy of the final product. Creams and lotions are often similar to one another, differing mainly in their viscosity. Both lotions and creams may be opaque, translucent or clear and often contain emulsifiers, solvents, and viscosity adjusting agents, as well as moisturizers, emollients, fragrances, dyes/colorants, preservatives and other active ingredients that increase or enhance the efficacy of the final product. Gels can be prepared with a range of viscosities, from thick or high viscosity to thin or low viscosity. These formulations, like those of lotions and creams, may also contain solvents, emulsifiers, moisturizers, emollients, fragrances, dyes/colorants, preservatives and other active ingredients that increase or enhance the efficacy of the final product. Liquids are thinner than creams, lotions, or gels, and often do not contain emulsifiers. Liquid topical products often contain solvents, emulsifiers, moisturizers, emollients, fragrances, dyes/colorants, preservatives and other active ingredients that increase or enhance the efficacy of the final product.

Emulsifiers for use in topical formulations include, but are not limited to, ionic emulsifiers, cetearyl alcohol, non-ionic emulsifiers like polyoxyethylene oleyl ether, PEG-40 stearate, ceteareth-12, ceteareth-20, ceteareth-30, ceteareth alcohol, PEG-100 stearate and glyceryl stearate. Suitable viscosity adjusting agents include, but are not limited to, protective colloids or nonionic gums such as hydroxvethylcellulose, xanthan gum, beeswax, paraffin, and cetyl palmitate. A gel composition may be formed by the addition of a gelling agent such as chitosan, methyl cellulose, ethyl cellulose, polyvinyl alcohol, polyquaterniums, hydroxyethylcellulose, hydroxypropylcellulose, hydroxypropylmethylcellulose, carbomer or ammoniated glycyrrhizinate. Suitable surfactants include, but are not limited to, nonionic, amphoteric, ionic and anionic surfactants. For example, one or more of dimethicone copolyol, polysorbate 20, polysorbate 40, polysorbate 60, polysorbate 80, lauramide DEA, cocamide DEA, and cocamide MEA, oleyl betaine, cocamidopropyl phosphatidyl PG-dimonium chloride, and ammonium laureth sulfate may be used within topical formulations.

Preservatives include, but are not limited to, antimicrobials such as methylparaben, propylparaben, sorbic acid, benzoic acid, and formaldehyde, as well as physical stabilizers and antioxidants such as vitamin E, sodium ascorbate/ascorbic acid and propyl gallate. Suitable moisturizers include, but are not limited to, lactic acid and other hydroxy acids and their salts, glycerine, propylene glycol, and butylene glycol. Suitable emollients include lanolin alcohol, lanolin, lanolin derivatives, cholesterol, petrolatum, isostearyl neopentanoate and mineral oils. Suitable fragrances and colours include, but are not limited to, FD&C Red No. 40 and FD&C Yellow No. 5. Other suitable additional ingredients that may be included in a topical formulation include, but are not limited to, abrasives, absorbents, anticaking agents, antifoaming agents, antistatic agents, astringents (such as witch hazel), alcohol and herbal extracts such as chamomile extract, binders/excipients, buffering agents, chelating agents, film forming agents, conditioning agents, propellants, opacifying agents, pH adjusters and protectants.

Typical modes of delivery for topical compositions include application using the fingers, application using a physical applicator such as a cloth, tissue, swab, stick or brush, spraying including mist, aerosol or foam spraying, dropper application, sprinkling, soaking, and rinsing. Controlled release vehicles can also be used, and compositions may be formulated for transdermal administration (for example, as a transdermal patch).

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions and particles of the invention may be administered orally, nasally, intravenously, intramuscularly, ocularly, transdermally, intraperitoneally, or subcutaneously. In one embodiment, the particles of the invention are administered intravenously.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S. P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

The effective amounts and method of administration of the present invention for modulation of an immune response can vary based on the individual, what condition is to be treated and other factors evident to one skilled in the art. Factors to be considered include route of administration and the number of doses to be administered.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. In some embodiments, the formulation provides a relatively constant level of modulator release. The amount of modulator contained within a sustained release formulation depends upon, for example, the site of implantation, the rate and expected duration of release and the nature of the condition to be treated or prevented.

In another embodiment there is provided a kit or article of manufacture including one or more compositions, peptides and/or pharmaceutical compositions as described above.

In other embodiments there is provided a kit for use in a therapeutic or prophylactic application mentioned above, the kit including:
a container holding a more compositions, peptides and/or pharmaceutical compositions as described herein:
a label or package insert with instructions for use.

In certain embodiments the kit may contain one or more further active principles or ingredients for treatment of an autoimmune disease, particularly a demyelination disorder such as multiple sclerosis.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat an inflammatory disease or condition described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a disorder or prevent a complication stemming from an autoimmune disease described herein. Alternatively, or additionally, the kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic, prophylactic or pharmaceutical composition. In one embodiment, the device is a syringe. The device may hold 1-2 mL of the therapeutic composition. The therapeutic or prophylactic composition may be provided in the device in a state that is ready for use or in a state requiring mixing or addition of further components.

### EXAMPLES

### Example 1—Methodology

Preparation of the MRI Imageable Silica Nanoparticles

MRI imageable Silica nanoparticles (SNPs) were prepared according to the modified Stober's method by hydrolysis and condensation of siloxane precursors-Tetraethylorthosilicate (TEOS) in the presence of ethanol and ammonia where doping elements for MRI is dissolved in ethanol. Briefly, 5 mL of TEOS was added to a solution containing 100 mL ethanol (96% v/v) containing 1-20 at % Fe²⁺/Fe³⁺ with respect to Si, and 4 mL 28% ammonia in water and ultrasonicated it for 5 minutes using a 3 mm probe at 30% amplitude (VCX 130, Sonics Vibra Cell). In case of porous silica preparation, a surfactant (1-2% CTAB or pluronic 123) is dissolved in ethanol prior to adding TEOS. The resultant solution was then vigorously stirred at 30° C. for 24 h to yield a colloidal solution. The solution formed were then separated by high-speed centrifugation and the silica particles were washed by 70% ethanol twice followed by washing with absolute ethanol to remove undesirable particles and finally resuspended in absolute ethanol. Subsequently, the resultant silica particles were washed with ethanol and dried at 60° C. under vacuum for further use. For creating mesoporosity, the CTAB Pluronic containing silica NPs are annealed at 400-600 degree Celsius for 5-10 hrs. The prepared silica nanoparticles were further functionalized to introduce amino groups on its surface. This was done by ultrasonicating ethanolic solution containing 400 mg silica nanoparticles along with 200 uL amino silane reagent for 5 minutes and vigorously stirring the solution for 5 h. The resultant solution were then washed 5 times with absolute ethanol followed by 2 times washing with deionized water to remove the unreacted reagents and finally redispersed it in water for further use. Characterization of the prepared NPs were done by Dynamic Light Scattering, Scanning Electron Microscopy, Transmission Electron Microscopy and magnetic resonance imaging to obtain the particle size, surface charge, particle uniformity and magneto resonance contrast studies.

Preparation of Peptide Conjugated Silica Nanoparticles

The various MOG peptides were conjugated to the functionalized silica nanoparticles using the EDC mediated chemistry. Briefly, MOG peptide dissolved in 0.1M MES buffer (pH 5.5) at a concentration of 1.1 mg/mL was added to amine functionalized Silica NPs dispersed in 0.1M MES buffer at the concentration of 2.08 mg/mL and stirred it for 5 min at low rpm. This was followed by the addition of EDC dissolved in 0.1 MES dropwise to the above solution and continued the reaction for 3 h in dark. The peptide conjugated NPs formed were separated by high-speed centrifugation and were washed twice with deionized water to remove unreacted reagents and the concentration of MOG peptides in the supernatant was measured using absorbance measurement in UV-visible spectroscopy. The peptide loaded on to the nanoparticles was calculated from the standard curve prepared using known amounts of MOG peptide and analyzed by measuring absorbance at 280 nm wavelength using UV-visible spectroscopy

EAE Induction and Scoring

EAE was induced in 10 week old female mice by subcutaneous injection of 100 μg recombinant mouse MOG (amino acid residues 1-117 of the mature protein, produced in-house as described Payne N L, et al. PLoS One. 2012; 7(6): e35093.) for NOD/Lt mice or 200 μg of the encephalitogenic peptide MOG35-55 (Chinapeptide) for C57Bl/6 mice emulsified in complete Freund's adjuvant (Sigma) supplemented with 400 mg Mycobacterium tuberculosis (BD) into both hind limb flanks. Mice immediately received an intraperitoneal injection of 350 ng pertussis toxin (List Biological Laboratories) and again 48 hr later. The mice were monitored daily, and clinical scores were assigned according to an arbitrary scale as follows: 0 normal, 1 loss of tail tone only, 2 weakness in one or two hind limbs and abnormal gait, 3 hind limb paralysis, 4 hind limb paralysis and fore limb weakness, and 5 dead. The mice were humanely killed by carbon dioxide asphyxiation upon reaching a score of 4 or at the completion of the experiment. The C57Bl/6 mouse model is a chronic progressive EAE model and the NOD/Lt is a relapsing remitting EAE model. Controls in both models displayed severe disease as the maximum average score of the mice reached at least 3 and close to 4, whereas maximum clinical scores of 2 represent a milder form of EAE.

Nanoparticle Delivery

Seven days and one day prior to EAE induction, nanoparticles coated with MOG37-52, scrambled MOG37-52, MOG44BF, ovalbumin or bear nanoparticles were injected intravenously into the tail vein of mice. Mice received the equivalent of 25 μg of peptide per injection in a volume of 150 μl. Myelin oligodendrocyte glycoprotein (MOG) derived peptides coupled to nanoparticles and used herein include:

MOG35-55
MEVGWYRSPFSRVVHLYRNGK
MOG37-52
VGWYRSPFSRVVHLYR
Scrambled MOG37-52
RSVLVPSRWRHFYGVY
MOG44BF
VGWYRSPβFSRVVHLYR

Histological Analysis

For histological analysis of CNS tissue, the brain and spinal cord were dissected from mice then fixed in 10% formalin (Sigma) for 24-48 h. Tissue was then processed and embedded in paraffin wax. Serial sections (5 μm) were cut from paraffin-embedded tissues and stained with hematoxylin and eosin, luxol fast blue and Bielschowsky silver impregnation to assess inflammation, demyelination and axonal damage, respectively. Sections were scored blind for semi-quantitative histological analysis of inflammation as follows: 0, no inflammation: 1, cellular infiltrate only in the perivascular areas and meninges; 2, mild cellular infiltrate in parenchyma: 3, moderate cellular infiltrate in parenchyma; 4, severe cellular infiltrate in parenchyma. Demyelination and axonal pathology were assessed by pale staining and scored blind as follows: 0, no demyelination/axonal pathology: 1, mild demyelination/axonal pathology; 2, moderate demyelination-axonal pathology; 3, severe demyelination/axonal pathology.

Proliferation Assay

Spleens were dissected from MOG T-cell receptor transgenic mice expressing the transgenic V3.2α/Vβ11 T-cell receptor specific for MOG35-55 peptide (MEVGWYRSPFSRVVHLYRNGK) in the context of 1-A^{b}. Single cell suspensions were prepared in complete RPMI medium containing 10% heat inactivated FBS, 2 mM L-glutamine, 100 U/ml penicillin, 100 μg/ml streptomycin, 50 μM 2-mercaptoethanol and 1 mM sodium pyruvate (Sigma). Following red blood cell lysis, splenocytes were seeded in triplicate in 96-well, flat bottom microtiter plates (Nunc) at a concentration of 2.5×10⁵ cells per well. Splenocytes were cultured in medium alone or in the presence of increasing doses of MOG35-55 or nanoparticles. Cells were incubated at 37° C. for 72 hr with the addition of 1 μCi/well [3H]-thymidine (Perkin Elmer) for the last 18 hr of culture. Cells were harvested onto filter mats (Perkin Elmer) and incorporated radioactive nucleic acids counted using a Top Count NXT Scintillation Counter (Packard Biosciences).

### Example 2—Results

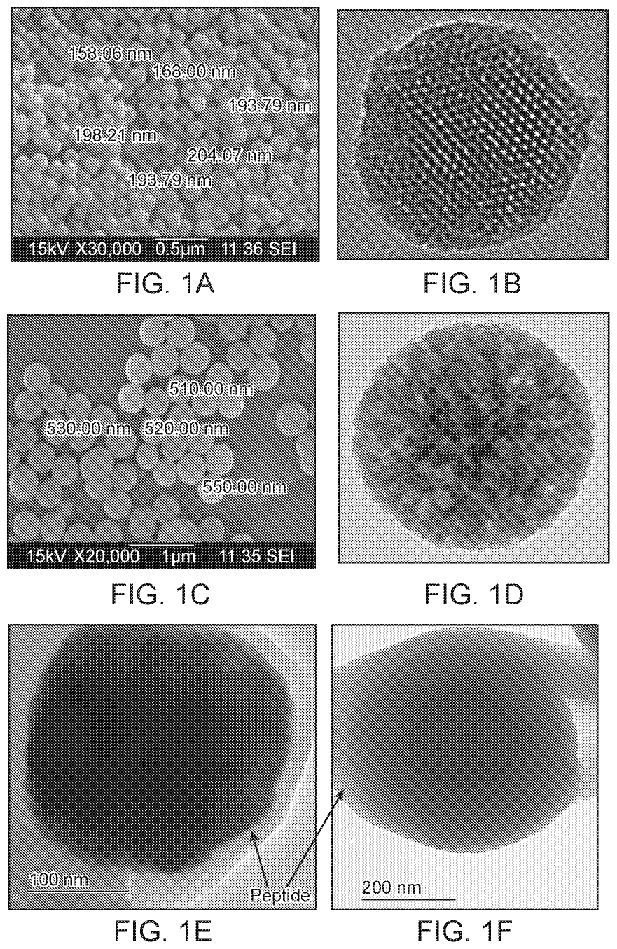

Two different sized silica NPs were synthesized as mentioned in the methodology. Characterization of NPs by SEM and TEM showed the uniform size distribution and porosity of NPs and the size were indeed 200 nm and 500 nm (FIGS. 1 A, 1 B. 1C, 1D). The surface charge of the prepared NPs were found to be negative (approximately −51 mV) for both the size of NPs (data not shown).

To attach the MOG peptides to the NPs, functionalization is done by introducing amine (—NH₂) groups over the surface as mentioned in the methodology, which was confirmed by the shift in the surface charge from negative to a positive value. Peptides were covalently attached to functionalized NPs by EDC chemistry as mentioned in the methodology. TEM images shown the presence of peptide coating over the surface of both 200 nm and 500 nm NPs.

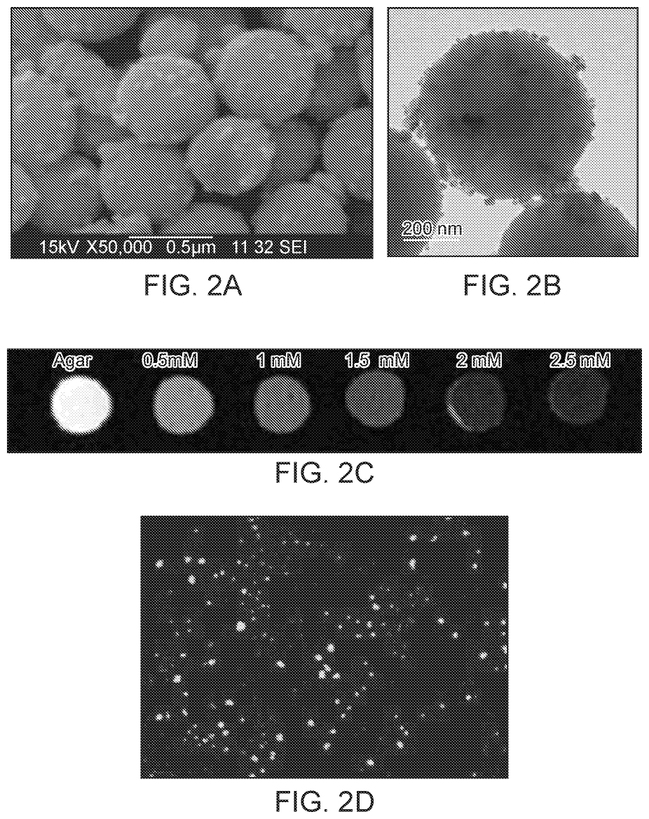

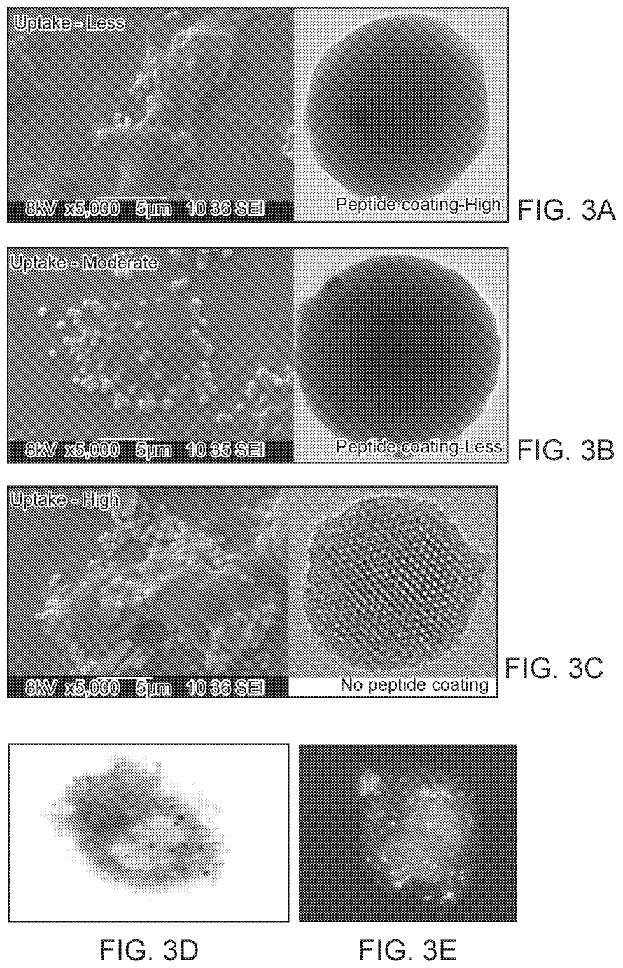

Since, the whole idea was to preferentially home in the peptide carrier nanoparticles to lymphoid organs like spleen and liver, the organs involved in induction of tolerance, we preferred the carrier particles to retain its property to be taken up by macrophages by phagocytosis, in spleen and liver. If the carrier particles are completely covered by thick peptide coating, the phagocytosis is not efficient whereas at optimum carrier to peptide ratio, some portion of the immunogenic nano-micro carrier particles are exposed to generate adequate immune response by way of macrophage-phagocytosis. To confirm the biodistribution of peptide coated NPs, we made the career NPs multi-functional by doping both the MRI contrast agent and a fluorescent dye on to the NPs, so that the particles can be tracked using MRI and fluorescence imaging respectively. FIG. 2A, 2B are SEM and TEM images of the multifuctional peptide coated career NPs. The synthesized multifuctional peptide coated career NPs were showing sufficient T2 contrast. For this, a phantom agar assay was done by diluting Fe doped silica (SiO₂) NPs in 1% agar phantoms with varying concentration of doped iron and imaging in MRI was taken. As shown in the FIG. 2C, there was an increase in the MR dark signal intensity of T2 contrast images when increasing the concentration from 0.5 mM to 2.5 mM. T2 weighted mapping studies were done to estimate the T2 relaxivity values of Fe doped SiO₂ NPs and plotted the graph to get relaxivity r2=75 mM⁻¹s⁻¹ (data not shown), which is sufficient enough to give a relevant in vivo T2 contrast. The fluorescent property of the multifuctional peptide coated career NPs were confirmed by fluorescent microscopy image that showed the green fluorescence (FIG. 3D), which is the fluorescence coming from the SiO₂ NPs.

Since macrophages play a crucial role in the entrapment of foreign bodies as well as in eliciting the innate and adaptive immunity, the uptake study of peptide coated multifuctional silica NPs on macrophages were studied using Prussian blue staining, fluorescent microscopy, scanning electron microscopy (SEM) etc.

All these uptake studies strongly confirm the major intracellular uptake seen in treated macrophages (FIG. 3). Notably, a huge uptake of peptide nanoparticles by the macrophage cells were seen, even at lower concentration (30 ug/mL) of particles, which are thought to be very crucial in the suppression of diasease in the EAE model of Multiple Sclerosis. Moreover, the optimum peptide conjugation is critical for maximum macrophage uptake of peptide-carrier conjugates. Decrease in the uptake of Peptide NPs by Macrophages was observed as the coating of peptide over NPs increases as shown in FIG. 3.

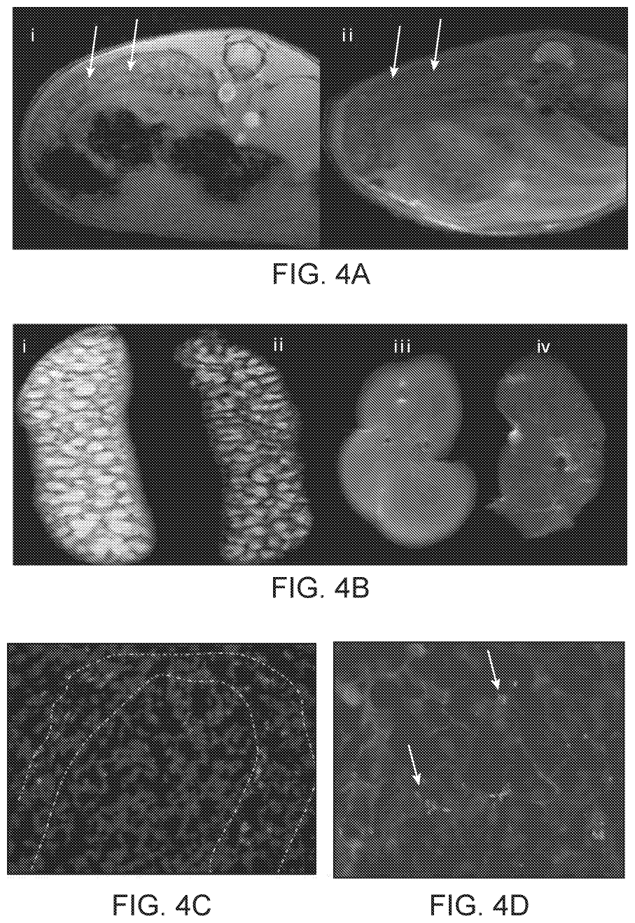

To confirm the targeting of engineered nanoparticles in lymphoid organs, multifunctional peptide coated career NPs were injected into C57BL/6 mice at a dose of 10 mg/kg to detect the biodistribution. It was found that spleen tissue was getting darkened during imaging after 2 h. Further, there was a notable darkening of spleen tissue during imaging after 4.5 h, which indicates the huge accumulation of peptide NPs in spleen (FIG. 4A). At this time point, animal was euthanized and organs like spleen and liver were collected for ex vivo imaging by MRI. Spleen and liver taken from untreated mice were used as control. As indicted from the FIG. 4B, there was a considerable difference in T₁ contrast of untreated mice (i) and multifunctional peptide coated career NPs injected mice (ii), which are clearly seen from the enhanced darkening of transverse-sectional ex vivo image of spleen.

Similarly, the liver of mice was also imaged by ex vivo using MRI, 4.5 h after the injection. Compared to liver of untreated mice, there was a significant darkening in the treated liver tissue which indicates the accumulation of NPs in liver also. Spleen and liver tissues were processed further for fluorescent microscopic imaging and found that the peptide coated NPs got accumulated in a specific concentric pattern in the spleen tissue, which may be the accumulation of particles over the marginal zone of white pulp region in spleen (FIG. 4A). Interestingly, the uptake of NPs in liver tissue was mainly seen in the kupffer cells whereas little presence of NPs are seen in hepatocytes.

Having achieved the targeting of peptide conjugated NPs, the efficacy of various MOG peptide conjugated NPs (MOG 37-52, MOG 44bF) were tested in the EAE models. Control treatments include bare NPs, scrambled MOG NPs and ovalbumin NPs. EAE models like the chronic progressive model (CPMS) in C57BL/6 mice and the relapsing remitting model (RRMS) in NOD/Lt mice were used for the study.

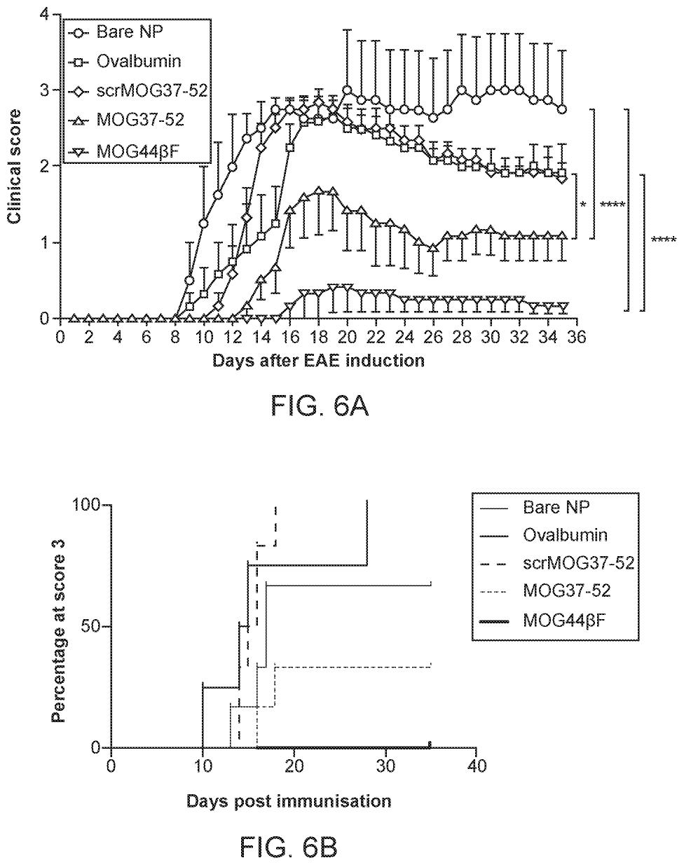

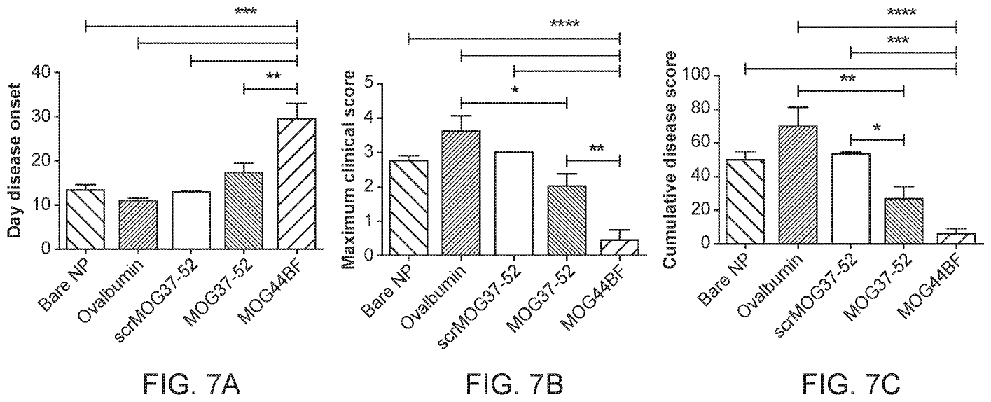

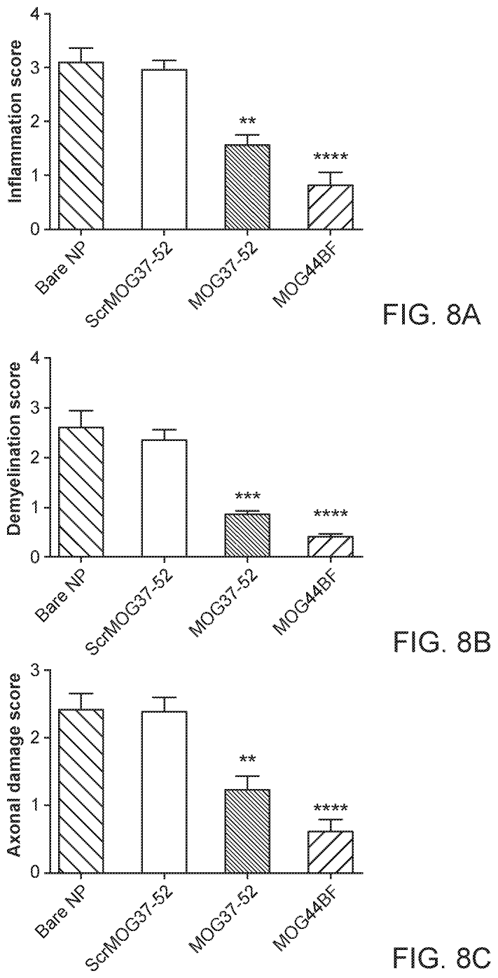

Treatment of C57BL/6 mice with 500 nm NPs coated with either MOG37-52 or MOG44βF prior to induction of EAE led to a significant reduction in the daily mean clinical score compared to controls (FIG. 6A) as well as a reduction in CNS inflammation, demyelination and axonal damage (FIG. 8). However MOG44βF coated nanoparticles produced a more significant therapeutic effect, with delayed onset (FIG. 7A) and greater attenuation of disease severity (FIG. 7B-C), compared to MOG37-52 coated NPs.

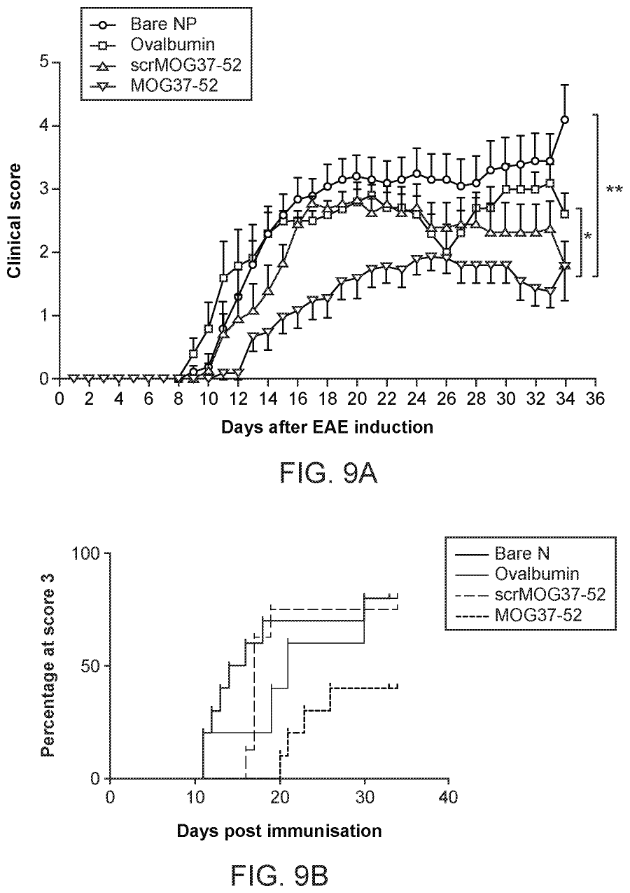

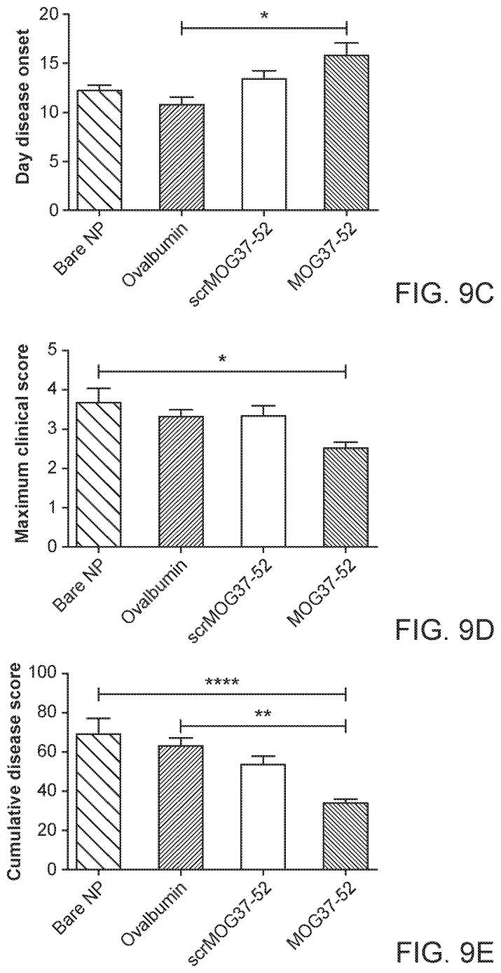

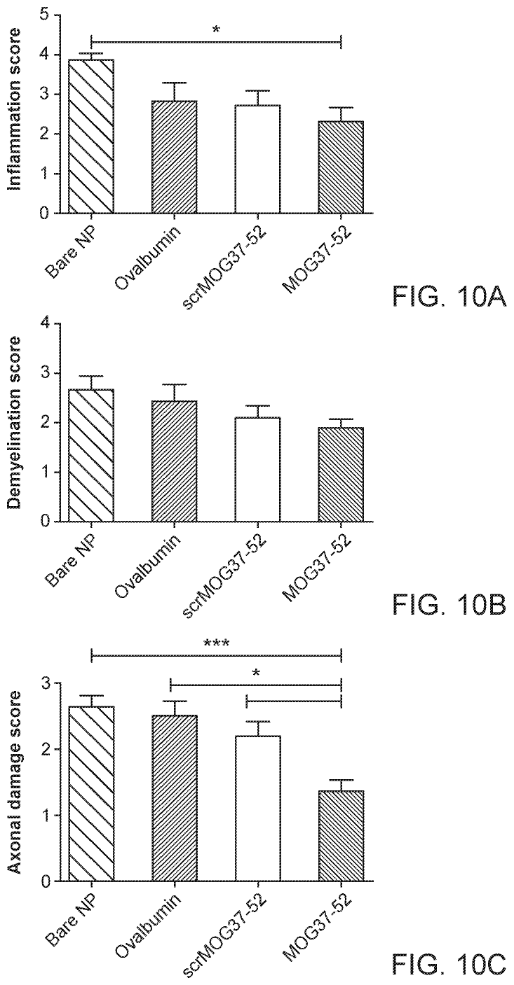

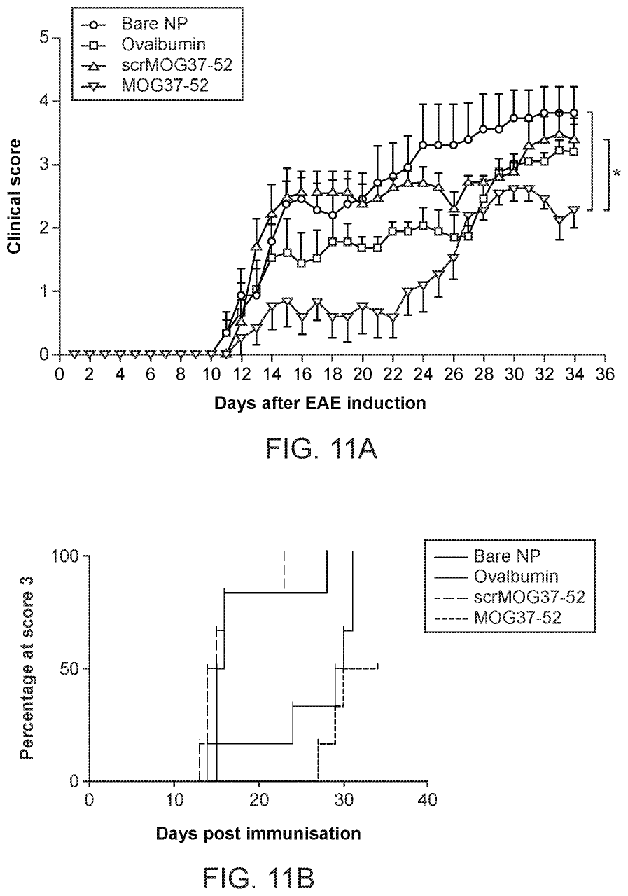

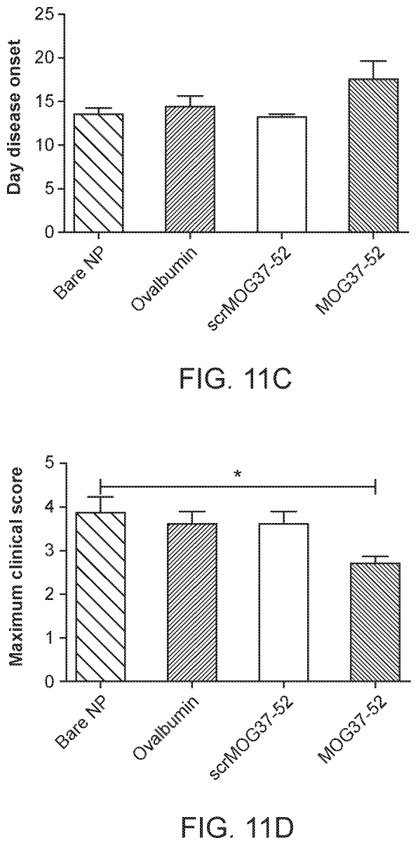

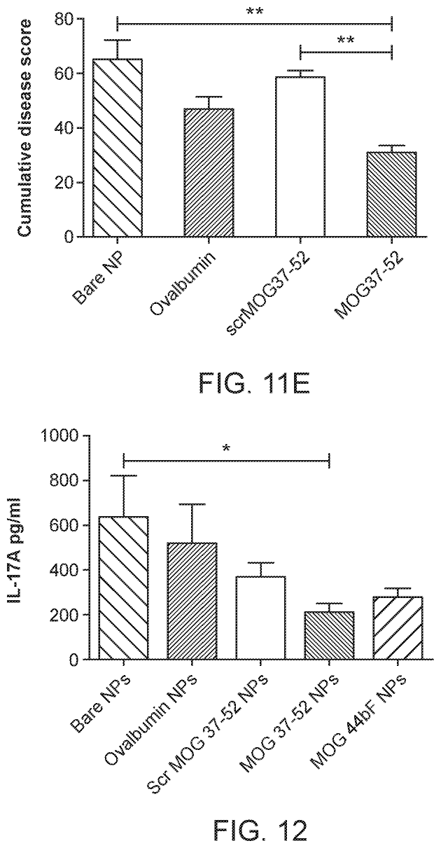

To compare MOG37-52 coated NPs to controls (scrambled, ovalbumin, bare particles) in two models with 200 nm NPs experiments in C57Bl/6 mice and NOD/Lt mice were performed as described herein. Treatment of C57BL/6 mice with MOG37-52 coated NPs had a significant effect on clinical disease (FIG. 9A; Table 2), but their overall effect on clinical disease was not as significant as that of the 500 nm NPs. Likewise, in NOD/Lt mice, 200 nm NPs coated with MOG37-52 significantly reduced disease severity (FIG. 11A Table 3).

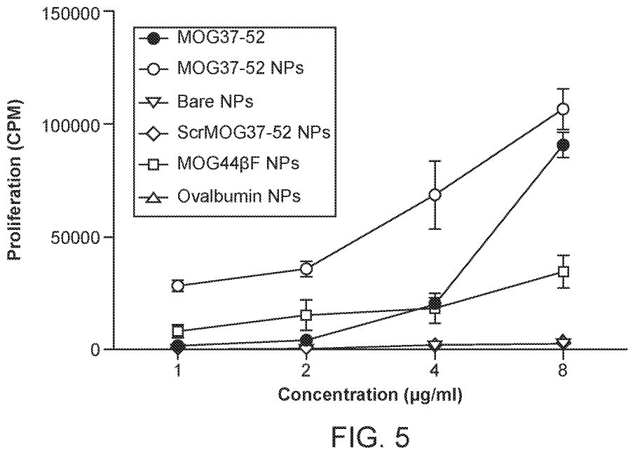

Stimulation of splenocytes from 2D2 mice with MOG35-55 induces a robust proliferation response (FIG. 5). 500 nm NPs coated with MOG37-52 were also capable of inducing a comparable proliferative response at 8 ug/ml (FIG. 5). Although MOG441F-coated NPs (FIG. 5) did not induce a robust proliferative response that was comparable to MOG37-52, it was higher than that induced by bare NPs, scrambled MOG37-52 and ovalbumin. Thus T-cells specific for MOG35-55 are capable of recognising MOG37-52 presented on the surface of the 500 nm NPs. There is some reactivity with MOG443F-coated NPs, however this is greatly reduced, likely due to the β-amino acid substitution impacting the interaction between the peptide and T-cell receptor. Amino acid substitutions at one or more residues in contact with the T-cell receptor or MHC molecules can alter T cell receptor-mediated effector functions of T cells, such as conferring T cell anergy, rendering them unresponsive to specific antigens, or altering downstream signalling pathways that change the function of T cells and the production of cytokines.

Stimulation of splenocytes from 2D2 mice with 200 nm NPs coated with MOG37-52 did not induce a proliferative response that was above background levels, with the exception of MOG37-52 coated NPs at the highest dose of 8 ug/ml (data not shown). However, this response was not comparable to the proliferative response of the native MOG37-52 peptide.

The reduced proliferative response of splenocytes to 200 nm nanoparticles is consistent with their overall reduced therapeutic effect in mice (FIG. 9, FIG. 11) compared to 500 nm nanoparticles (FIG. 6). The results here are consistent with previous studies showing that 500 nm NPs achieve more optimal immune responses than 200 nm NPs. Alternatively, the density of the peptide on the surface of the NPs may also be important. It is possible that the amount of peptide on the surface of the 200 nm NPs was too high, leading to stearic hindrance, whereby the higher peptide density would prevent the T-cell receptor from binding to the peptide on the nanoparticle.

EAE was induced in NOD/Lt mice with recombinant mouse MOG protein (rmMOG, amino acid residues 1-117). Unlike EAE induced with MOG35-55 peptide, rmMOG-induced EAE results in the activation of MOG-specific B-cells, allowing B-cells to process and present antigen to generate pathogenic T-cells as well as promoting the developing of antibodies recognising the native MOG protein (Weber M S et al. Ann Neurol. 2010; 68(3):369-83). Thus, the differences in the immunopathogenesis of MOG protein-induced EAE compared to that induced by MOG35-55 peptide may also impact the efficacy of the nanoparticles in NOD/Lt mice and a different size nanoparticle may be required to target B-cell responses.

TABLE 1
Clinical and histological features of C57Bl/6 EAE mice injected with 500 nm silica peptide nanoparticles.

| | MOG37-52 | MOG44BF | ScrMOG37-52 | Ova | Bear NP |
|---|---|---|---|---|---|
| Disease incidence | 6/6 | 2/6 | 6/6 | 4/4 | 6/6 |
| Mortality | 0/6 | 0/6 | 0/6 | 1/4 | 0/6 |
| Day disease onset | 17.17 ± 2.151 | 29.17 ± 3.710⁺⁺ ^{### *** ∧∧∧} | 12.67 ± 0.4216 | 10.75 ± 0.8539 | 13.33 ± 1.145 |
| Maximum score | 2.00 ± 0.3873^{*} | 0.4167 ± 0.3270⁺⁺ ^{###} ^{*** ∧∧∧} | 3.00 ± 0.0000 | 3.625 ± 0.4732 | 2.75 ± 0.1708 |
| Cumulative score | 26.00 ± 8.039^{#} ^{**} | 5.417 ± 3.967^{###} ^{**** ∧∧∧} | 52.5 ± 2.053 | 69.38 ± 11.77 | 49.25 ± 5.829 |
| Score at termination | 1.083 ± 0.3270^{*} | 0.1667 ± 0.1054^{*** #} ^{∧} | 1.833 ± 0.2108 | 2.75 ± 0.7773 | 1.917 ± 0.3745 |
| Inflammation score | 1.542 ± 0.2275^{##} ^{* ∧∧} | 0.7917 ± 0.2694^{### *** ∧∧∧∧} | 2.917 ± 0.2007 | 2.833 ± 0.1667 | 3.083 ± 0.2713 |
| Demyelination score | 0.833 ± 0.8333^{###} ^{** ∧∧∧∧} | 0.375 ± 0.08539^{### *** ∧∧∧∧} | 2.333 ± 0.2108 | 2.333 ± 0.1667 | 2.583 ± 0.3516 |
| Axonal damage | 1.208 ± 0.2275^{##} ^{∧∧} | 0.5833 ± 0.2007^{####** ∧∧∧∧} | 2.367 ± 0.2472 | 2.333 ± 0.1667 | 2.417 ± 0.2386 |

Data are expressed as either a fraction of the total number examined or the mean ± SEM
^{#}P < 0.05, ^{##}P < 0.01, ^{###}P < 0.001, ^{####}P < 0.0001 versus ScrMOG37-52 NPs, ^{∧}P < 0.05, ^{∧∧}P < 0.01, ^{∧∧∧}P < 0.001, ^{∧∧∧∧}P < 0.0001, versus Bare NPs, ^{*}P < 0.05, ^{**}P < 0.01, ^{***}P < 0.001, ^{****}P < 0.0001 versus ova NPs, ⁺⁺P < 0.01 versus MOG37-52 NPs, ANOVA

TABLE 2
Clinical and histological features of C57Bl/6 EAE mice injected with 200 nm silica peptide nanoparticles.

| | MOG37-52 | ScrMOG37-52 | Ova | Bear NP |
|---|---|---|---|---|
| Disease incidence | 10/10 | 8/8 | 5/5 | 10/10 |
| Mortality | 0/10 | 0/8 | 0/5 | 2/10 |
| Day disease onset | 15.70 ± 1.334^{*} | 13.38 ± 0.9051 | 10.60 ± 0.9274 | 12.20 ± 0.5333 |
| Maximum score | 2.50 ± 0.1667^{∧} | 3.313 ± 0.2825 | 3.3 ± 0.2000 | 3.65 ± 0.3877 |
| Cumulative score | 33.05 ± 2.086^{** ∧∧∧∧} | 52.50 ± 4.621 | 62.00 ± 4.987 | 68.35 ± 8.089 |
| Score at termination | 1.45 ± 0.2930^{∧∧∧} | 2.313 ± 0.4525 | 2.60 ± 0.3317 | 3.50 ± 0.415 |
| Inflammation score | 2.275 ± 0.3772^{∧} | 2.688 ± 0.3889 | 2.80 ± 0.4899 | 3.857 ± 0.1429 |
| Demyelination score | 1.85 ± 0.2242 | 2.063 ± 0.2745 | 2.40 ± 0.3674 | 2.643 ± 0.2608 |
| Axonal damage | 1.35 ± 0.1833^{* #} ^{∧∧∧} | 2.188 ± 0.2302 | 2.50 ± 0.2236 | 2.643 ± 0.1798 |

Data are expressed as either a fraction of the total number examined or the mean ± SEM
^{*}P < 0.05, ^{**}P < 0.01 versus ova NPs, ^{#}P < 0.05 versus ScrMOG37-52 NPs, ^{∧}P < 0.05, ^{∧∧}P < 0.01, ^{∧∧∧}P < 0.001, ^{∧∧∧∧}P < 0.0001, versus Bare NPs, ANOVA

TABLE 3
Clinical and histological features of NOD/Lt EAE mice injected with 200 nm silica peptide nanoparticles.

| | MOG37-52 | ScrMOG37-77 | Ova | Bear NP |
|---|---|---|---|---|
| Disease incidence | 6/6 | 6/6 | 6/6 | 6/6 |
| Mortality | 0/6 | 0/6 | 1/6 | 2/6 |
| Day disease onset | 17.33 ± 2.23 | 13.00 ± 0.4472 | 14.17 ± 1.327 | 13.33 ± 0.8819 |
| Maximum score | 2.667 ± 0.1667^{∧} | 3.583 ± 0.3005 | 3.583 ± 0.3005 | 3.833 ± 0.3801 |
| Cumulative score | 30.50 ± 3.266^{∧∧} ^{##} | 58.5 ± 2.772 | 46.75 ± 4.656 | 64.83 ± 7.524 |
| Score at termination | 2.25 ± 0.2814^{∧} | 3.333 ± 0.3333 | 3.167 ± 0.4014 | 3.75 ± 0.4031 |
| Inflammation score | 4.167 ± 0.1667 | 3.583 ± 0.2713 | 4.00 ± 0.00 | 3.25 ± 0.433 |
| Demyelination score | 2.917 ± 0.0833 | 2.583 ± 0.2007 | 2.60 ± 0.1871 | 1.875 ± 0.5154 |
| Axonal damage | 2.833 ± 0.1667 | 2.583 ± 0.2007 | 2.60 ± 0.1871 | 1.75 ± 0.4787 |

Data are expressed as either a fraction of the total number examined or the mean ± SEM
^{##}P < 0.01 versus ScrMOG37-52 NPs, ^{∧}P < 0.05, ^{∧∧}P < 0.01 versus Bare NPs, ANOVA

Relevance of MOG-EAE to Human Disease

EAE can be induced in susceptible mouse strains by immunisation with myelin proteins or encephalitogenic peptides in adjuvant to induce a T-cell mediated autoimmune disease targeting the CNS. EAE mouse models mimic many of the clinical and pathological features of MS and have contributed to our understanding of MS pathogenesis and the development of therapeutics. Although initial studies utilised the major myelin proteins MBP and PLP for EAE induction, MOG has emerged as an important target antigen not only in MS but other CNS demyelinating diseases including optic neuritis, acute disseminated encephalomyelitis, neuromyelitis optica spectrum disorders and transverse myelitis.

MOG is a 218 amino acid long Type I transmembrane protein that is found only in mammals, expressed exclusively in the central nervous system and is highly conserved across species. MOG is located on the outer layer of the myelin sheath and thus exposed to the immune system, with residues 35-55 located in a protruding loop.

T-cells from MS patients display predominant reactivity to native MOG, and unlike antibodies against MBP and PLP, anti-MOG antibodies display demyelinating activity in vivo and in vitro. Autoantibodies with demyelinating capacity recognize discontinuous/conformational epitopes of MOG. MOG autoantibodies can be detected in the sera and CSF of patients with MS, however they can be detected in healthy individuals as well (up to 30% in one study). It is likely that there is a subgroup of MS patients in which MOG antibodies are pathogenic while other MOG antibodies observed in patients and healthy controls are not pathogenic Autoantibodies recognizing conformational epitopes of MOG are found in 20-40% of children with demyelinating diseases.

MOG is the only myelin antigen that produces both an encephalitogenic T-cell response and autoantibody response in animal models of MS. Approximately 30% of 2D2 transgenic mice with CD4 T-cells specific for MOG35-55 develop spontaneous optic neuritis and 5% develop a spontaneous MS-like disease. Approximately 60% of mice in which T and B-cells are specific for MOG develop spontaneous MS-like disease. Immunisation of C57BL/6 with MOG35-55 induces a chronic progressive CD4 T-cell mediated disease that has become the gold standard for analysing the immunopathogenic mechanisms associated with MS as well as for assessing the efficacy of new therapeutics. The disease presents as ascending paralysis, with mice initially displaying loss of tail tone approximately 10 days after immunisation. This is followed by hind limb weakness and then hind limb paralysis.

Approximately 85% of MS patients display a relapsing-remitting disease (RR-MS) characterised by period of neurological deficits followed by partial or complete recovery. Over time, these functional deficits will accumulate without remission and this is described as the secondary progressive phase (SP-MS). On average RR-MS patients will develop permanent neurological disability 10 years after disease onset. MS patients require walking assistance 15-28 years after disease onset. A small proportion of patients (up to 15%) display a primary progressive clinical course (PP-MS), with increasing neurological deficits from onset with no remission.

It will be understood that the invention disclosed and defined in this specification extends to all alternative combinations of two or more of the individual features mentioned or evident from the text or drawings. All of these different combinations constitute various alternative aspects of the invention.

While the invention has been disclosed with reference to certain embodiments, it will be understood by those skilled in the art that various changes may be made and equivalents may be substituted without departing from the scope of the invention. In addition, many modifications may be made to adapt to a particular situation or material the teachings of the invention without departing from its scope.

What is claimed is:

1. A composition comprising: a modified peptide antigen comprising at least one beta (β) amino acid coupled, complexed or encapsulated to a carrier nano- or micro-particle of silica, wherein said nanoparticles are configured to be specifically phagocytosed by macrophages in liver, spleen and lymph nodes, and imagable by non-invasive method such as magnetic resonance imaging.

2. A composition according to claim 1, wherein the modified peptide antigen causes immunosuppression in autoreactive disease models at concentration of 10-50 times lower compared to corresponding free peptide.

3. A composition according to claim 1, wherein the β amino acid is present or introduced at a position in the peptide antigen where the homologous α (alpha) aminoacid in the peptide antigen interacts with a T cell receptor or MHC molecule.

4. A composition according to claim 1, wherein the peptide antigen is derived from myelin oligodendrocyte glycoprotein (MOG), myelin basic protein (MBP) or proteolipid protein (PLP).

5. A composition comprising: a myelin oligodendrocyte glycoprotein (MOG) peptide coupled, complexed or encapsulated to a carrier particle, wherein the peptide comprises an amino acid sequence having at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 1, wherein the amino acid residue at, or equivalent to:
position 38 in SEQ ID NO: 1 is not an αGly;
position 39 in SEQ ID NO: 1 is not an αLys;
position 40 in SEQ ID NO: 1 is not an αTyr;
position 41 in SEQ ID NO: 1 is not an αArg;
position 43 in SEQ ID NO: 1 is not an αPro;
position 44 in SEQ ID NO: 1 is not an αPhe;
position 45 in SEQ ID NO: 1 is not an αSer;
position 46 in SEQ ID NO: 1 is not an αArg;
position 47 in SEQ ID NO: 1 is not an αVal; or
position 48 in SEQ ID NO: 1 is not an αVal.

6. A composition according to claim 5, wherein the amino acid residue is a non-conservative substitution relative to the amino acid that occurs in that position in SEQ ID NO: 1.

7. A composition according to claim 5, wherein the amino acid residue is alanine or a β amino acid.

8. A composition according to claim 7, wherein the amino acid residue is a β amino acid version of the amino acid that occurs at that position in SEQ ID NO: 1.

9. A composition according to claim 7, wherein the amino acid residue at position 44 is an alanine.

10. A composition according to claim 9, wherein the amino acid residue at position 44 is a β-alanine.

11. A composition according to claim 8, wherein the amino acid residue at position 44 is a β-phenylalanine.

12. A composition comprising: a myelin oligodendrocyte glycoprotein (MOG) peptide coupled, complexed or encapsulated to a carrier particle, wherein the peptide comprises an amino acid sequence having at least 60%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity with SEQ ID NO: 7, wherein the amino acid residue at, or equivalent to position 119 in SEQ ID NO: 7 is not an αPhe.

13. A composition according to claim 12, wherein the amino acid at position 119 is α-alanine, β-phenylalanine or β-alanine.

14. A composition according to claim 1, wherein the carrier particle comprises solid or porous silica and the particle diameter is 10-5000 nm.

15. A composition according to claim 1, wherein the carrier particle is formed by solid or porous silica of spherical, rectangular, triangular, needular, prismatic, rod shape, or any combination thereof.

16. A composition according to claim 1, wherein the carrier particle is doped with Fe, Gd or Mn at a concentration of 0.1-50 wt % with respect to Si.

17. A composition according to claim 1, wherein the minimum weight ratio of peptide to nanoparticle or microparticle that is critical for the specific uptake in liver, spleen and lymph node residing macrophage is about 0.01.

18. A composition according to claim 1, wherein, at optimum concentration of peptide-nanoparticle conjugation and macrophage uptake, the cells produce reduced levels of one of the main pro-inflammatory cytokine, IL-17, that causes autoreactivity.

19. A composition according to claim 1, wherein the carrier particle is carboxy functionalized by EDTA (Ethylene diamine tetra acetic acid), DTPA (Diethylene triamine penta acetic acid), Succinic acid, malonic acid, glutaric acid, adipic acid, pimelic acid or hyaluronic acid.

20. A composition according to claim 1, wherein the carrier particle is amino functionalized by amino propyl triethoxy silanes, amino propyl trimethoxy silane, polyethylene imine, protamine sulfate, poly-L-Lysine or arginine.

21. A composition according to claim 19, wherein the carboxy functionalized carrier particle is attached to a linker made of amino group provided by amino propyl triethoxy silanes, amino propyl trimethoxy silane, polyethylene imine, protamine sulfate, poly-L-Lysine or arginine.

22. A composition according to claim 20, wherein the amino functionalized carrier particle is attached to a linker made of carboxyl group provided by EDTA, DTPA, Succinic acid, malonic acid, glutaric acid, adipic acid, pimelic acid or hyaluronic acid by forming an amide bond, or by electrostatic interactions.

23. A method of treating, preventing, delaying the onset, or reducing the severity of an autoimmune disease comprising: administering to a subject in need thereof a composition comprising a modified peptide antigen comprising at least one beta (β) amino acid coupled, complexed or encapsulated to a carrier nano- or micro-particle of silica, wherein said nanoparticles are configured to be specifically phagocytosed by macrophages in liver, spleen and lymph nodes, and imagable by non-invasive method such as magnetic resonance imaging.

24. A method according to claim 23, further comprising the step of identifying a subject at risk of developing an autoimmune disease.

25. A composition according to claim 5, wherein the carrier particle comprises solid or porous silica and the particle diameter is 10-5000 nm.

26. A composition according to claim 5, wherein the carrier particle is formed by solid or porous silica of spherical, rectangular, triangular, needular, prismatic, rod shape, or any combination thereof.

27. A composition according to claim 5, wherein the carrier particle is doped with Fe, Gd or Mn at a concentration of 0.1-50 wt % with respect to Si.

28. A composition according to claim 5, wherein the minimum weight ratio of peptide to nanoparticle or microparticle that is critical for the specific uptake in liver, spleen and lymph node residing macrophage is about 0.01.

29. A composition according to claim 5, wherein, at optimum concentration of peptide-nanoparticle conjugation and macrophage uptake, the cells produce reduced levels of one of the main pro-inflammatory cytokine, IL-17, that causes autoreactivity.

30. A composition according to claim 5, wherein the carrier particle is carboxy functionalized by EDTA (Ethylene diamine tetra acetic acid), DTPA (Diethylene triamine penta acetic acid), Succinic acid, malonic acid, glutaric acid, adipic acid, pimelic acid or hyaluronic acid.

31. A composition according to claim 5, wherein the carrier particle is amino functionalized by amino propyl triethoxy silanes, amino propyl trimethoxy silane, polyethylene imine, protamine sulfate, poly-L-Lysine or arginine.

32. A composition according to claim 30, wherein the carboxy functionalized carrier particle is attached to a linker made of amino group provided by amino propyl triethoxy silanes, amino propyl trimethoxy silane, polyethylene imine, protamine sulfate, poly-L-Lysine or arginine.

33. A composition according to claim 31, wherein the amino functionalized carrier particle is attached to a linker made of carboxyl group provided by EDTA, DTPA, Succinic acid, malonic acid, glutaric acid, adipic acid, pimelic acid or hyaluronic acid by forming an amide bond, or by electrostatic interactions.

34. A composition according to claim 12, wherein the carrier particle comprises solid or porous silica and the particle diameter is 10-5000 nm.

35. A composition according to claim 5, wherein the carrier particle is formed by solid or porous silica of spherical, rectangular, triangular, needular, prismatic, rod shape, or any combination thereof.

36. A composition according to claim 12, wherein the carrier particle is doped with Fe, Gd or Mn at a concentration of 0.1-50 wt % with respect to Si.

37. A composition according to claim 12, wherein the minimum weight ratio of peptide to nanoparticle or microparticle that is critical for the specific uptake in liver, spleen and lymph node residing macrophage is about 0.01.

38. A composition according to claim 12, wherein, at optimum concentration of peptide-nanoparticle conjugation and macrophage uptake, the cells produce reduced levels of one of the main pro-inflammatory cytokine, IL-17, that causes autoreactivity.

39. A composition according to claim 12, wherein the carrier particle is carboxy functionalized by EDTA (Ethylene diamine tetra acetic acid), DTPA (Diethylene triamine penta acetic acid), Succinic acid, malonic acid, glutaric acid, adipic acid, pimelic acid or hyaluronic acid.

40. A composition according to claim 12, wherein the carrier particle is amino functionalized by amino propyl triethoxy silanes, amino propyl trimethoxy silane, polyethylene imine, protamine sulfate, poly-L-Lysine or arginine.

41. A composition according to claim 39, wherein the carboxy functionalized carrier particle is attached to a linker made of amino group provided by amino propyl triethoxy silanes, amino propyl trimethoxy silane, polyethylene imine, protamine sulfate, poly-L-Lysine or arginine.

42. A composition according to claim 40, wherein the amino functionalized carrier particle is attached to a linker made of carboxyl group provided by EDTA, DTPA, Succinic acid, malonic acid, glutaric acid, adipic acid, pimelic acid or hyaluronic acid by forming an amide bond, or by electrostatic interactions.


SEQUENCE LISTING (10 sequences)

SEQ ID NO: 1 - protein, 16 aa, Homo sapiens
VGWYRPPFSRVVHLYR

SEQ ID NO: 2 - protein, 16 aa, Homo sapiens
  MOD_RES (8)..(8): beta phenylalanine
VGWYRPPXSRVVHLYR

SEQ ID NO: 3 - protein, 21 aa, Homo sapiens
MEVGWYRPPFSRVVHLYRNGK

SEQ ID NO: 4 - protein, 16 aa, Mus musculus
VGWYRSPFSRVVHLYR

SEQ ID NO: 5 - protein, 16 aa, Mus musculus
  MOD_RES (8)..(8): beta phenylalanine
VGWYRSPXSRVVHLYR

SEQ ID NO: 6 - protein, 21 aa, Mus musculus
MEVGWYRSPFSRVVHLYRNGK

SEQ ID NO: 7 - protein, 14 aa, Homo sapiens
FYWVNPGVLTLIAL

SEQ ID NO: 8 - protein, 224 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFESFGVLGPQVKEPKKTGQFLEELRNPF

SEQ ID NO: 9 - protein, 13 aa, Homo sapiens
VHFFKNIVTPRTP

SEQ ID NO: 10 - protein, 13 aa, Homo sapiens
HSLGKWLGHPDKF